(19) United States
(12) Patent Application Publication
(10) Pub. No.: US 2004/0247605 A1
(43) Pub. Date: Dec. 9, 2004

(54) Wall teichoic acid as a target for anti-staphylococcal therapies and vaccines

(76) Inventors: John Fitzgerald Kokai-Kun, Frederick, MD (US); Andreas Peschel, Tubingen (DE); Christopher Weidenmaier, Tubingen (DE); Sascha A. Kristian, Duesseldorf (DE)

Correspondence Address: FINNEGAN, HENDERSON, FARABOW,, GARRETT & DUNNER, L.L.P., 1300 I Street, N.W., Washington, DC, 20005-3315, US

(21) Appl. No.: 10/724,194

(22) Filed: Dec. 1, 2003

Related U.S. Application Data
(60) Provisional application No. 60/430,225, filed on Dec. 2, 2002.

(51) Int. Cl. A61K 39/00; A61K 39/38

(52) U.S. Cl. 424/184.1

(57) ABSTRACT

This invention provides vaccines comprising staphylococcal wall teichoic acid (WTA); vaccines comprising antibodies that specifically bind WTA; staphylococcal organisms deficient in WTA; and methods of treating patients suspected of having a staphylococcal infection.

### CROSS-REFERENCE TO RELATED APPLICATIONS

This application is based on and claims the benefit of U.S. Provisional Application Ser. No. 60/430,225, filed Dec. 2, 2002. The entire disclosure of this provisional application is relied upon and incorporated by reference herein.

### DESCRIPTION OF THE INVENTION

1. Field of the Invention

This invention in the fields of immunology and infectious diseases relates to antibodies that are specific for Gram positive bacteria, particularly to bacteria that bear wall teichoic acid (WTA) on their surfaces. The invention includes polyclonal antibodies. The invention also includes monoclonal, chimeric, and humanized antibodies, as well as fragments, regions and derivatives thereof. This invention further relates to Gram positive bacteria deficient in WTA. This invention also relates to vaccines comprised of WTA and to vaccines comprised of antibodies that are specific for WTA. In addition to therapeutic applications, the antibodies of the invention may also be used for diagnostic and prophylactic applications.

2. Background of the Invention

The search for agents to combat bacterial infections has been long and arduous. The development of antibiotics has brought us from the time when sepsis associated with amputation was associated with a 50 percent mortality rate. Today's challenge, however, is the increasing development of bacteria that are resistant to antibiotics, such as members of the genera Staphylococcus.

Staphylococci are particularly worrisome because they commonly colonize humans and animals and are an important cause of human morbidity and mortality. Because of their prevalence on the skin and mucosal linings, staphylococci are ideally situated to produce both localized and systemic infections. Of the staphylococci, both S. aureus, a coagulase positive bacteria, and S. epidermidis, a coagulase negative species, are the most problematic.

In fact, S. aureus is the most virulent staphylococcus, producing severe and often fatal disease in both normal and immunocompromised patients. S. aureus, a highly pathogenic species of staphylococci, is often found in the anterior nares of humans as a primary ecological niche. It is well documented that S. aureus nasal colonization is a significant risk factor for contracting S. aureus infection and a means for community spread of multi-drug resistance S. aureus.

S. epidermidis has become one of the major causes of nosocomial (hospital acquired) infection in patients with impaired immune responses or those whose treatments involve the placement of foreign objects into the body, such as patients who receive continuous ambulatory peritoneal dialysis and patients receiving parenteral nutrition through central venous catheters (64). Indeed, S. epidermidis is now recognized as a common cause of neonatal nosocomial sepsis, and infections frequently occur in premature infants that have received parenteral nutrition. Moreover, in recent years, the involvement of S. epidermidis in neonatal infection has increased dramatically. Indeed, for every 10 babies diagnosed with bacterial sepsis seven or more days after birth (indicative of post-partum bacterial exposure), six of those are infected with S. epidermidis. Untreated, Staphylococcus infections in newborns can result in multiple organ failure and death in two to three days. Antibiotics are only partially effective and, unfortunately, the rise in multiply drug resistant strains of Staphylococcus renders antibiotic treatments less and less effective.

The problems of antibiotic resistance are so significant that they have reached the lay press. See, e.g., The Washington Post “Microbe in Hospital Infections Show Resistance to Antibiotics,” May 29, 1997; The Washington Times, “Deadly bacteria outwits antibiotics,” May 29, 1997. And this concern is borne out by the scientific literature. See L. Garrett, The Coming Plague, “The Revenge of the Germs or Just Keep Inventing New Drugs” Ch. 13, pgs. 411-456, Farrar, Straus and Giroux, N.Y., Eds. (1994). In one study, the majority of staphylococci isolated from blood cultures of septic infants were resistant to multiple antibiotics (33). Another study describes methicillin-resistant S. aureus (75). There is no doubt that the emergence of antibiotic resistance among clinical isolates is making treatment difficult (47).

As discussed above, staphylococcal infections continue to be a major health problem and with the emerging resistance of staphylococci to most available antibiotics, alternative approaches are clearly needed. At least three strategies present themselves for combating staphylococcal infections. First, the body can be primed to fight off infections through its own immune system, and this may be accomplished through vaccines. Second, the infectious process of the pathogen may be interfered with at some stage of the process. For example, the initial adherence/colonization by the pathogen may be prevented so that the infectious process does not even have a chance to begin. Third, an established infection can be treated by various means, including but not limited to antibiotics, to eliminate the pathogen. However, emerging resistance to drug-based treatments is becoming less effective. Strategies to develop vaccines or to prevent adherence/colonization remain viable alternatives, as indicated below.

Vaccines: Antibodies may be administered directly to a patient. Alternatively, antibodies may be induced by vaccinating a patient with an antigen composition that stimulates production of antibodies that specifically react with a bacterium or a bacterial component. Antibodies protect against bacterial attack by recognizing and binding to antigens on the bacteria to thereby facilitate the removal or “clearance” of the bacteria by a process called phagocytosis, wherein phagocytic cells (predominantly neutrophils and macrophages) identify, engulf, and subsequently destroy the invading bacteria. Antibodies may also have the effect of interfering with the infectious process by blocking some important host/pathogen interaction. However, bacteria have developed mechanisms to avoid phagocytosis, such as the production of a “capsule” to which phagocytes cannot adhere or the production of toxins that actually poison the encroaching phagocytes. Antibodies overcome these defenses by, for example, binding to the toxins to thereby neutralize them. More significantly, antibodies may themselves bind to the capsule to coat it, in a process called opsonization, and thus make the bacteria extremely attractive to phagocytes and to enhance their rate of clearance from the bloodstream.

Confounding the use of passively administered antibodies or antibodies induced by vaccination, however, are conflicting reports in the literature. For example, the immunization studies of Fattom et al. demonstrated that opsonization of S. epidermidis was related to the specific capsule type, as with S. aureus and other encapsulated Gram positive bacteria such as Streptococcus pneumonia (29). In another study, Timmerman et al. identified a surface protein of S. epidermidis that induced opsonic monoclonal antibodies (88). Timmerman et al. also identified other monoclonal antibodies that bound to non-homologous S. epidermidis strains, but only the monoclonal antibody produced to the homologous strain was opsonic, thus opsonization was enhanced only to the homologous strain but not to heterologous strains. Accordingly, based on the studies of Fattom et al., and Timmerman et al., and others in the field (and in contrast to our laboratory's studies as set forth in U.S. Pat. Nos. 5,571,511 and 5,955,074), one would not expect that an antibody that is broadly reactive to multiple strains of S. epidermidis and to S. aureus would have opsonic activity against each strain. This is particularly true for antibodies that bind to both coagulase positive and coagulase negative staphylococci.

Further exacerbating the problem, the role of the common surface antigens on staphylococci has been unclear. For example, while lipoteichoic acid and teichoic acid make up the majority of the cell wall of S. aureus, there was no prior appreciation that antibodies to lipoteichoic acid and teichoic acid could be protective. Indeed, anti-teichoic acid antibodies have been often used as controls. For example, Fattom et al. examined the opsonic activity of antibodies induced against a type-specific capsular polysaccharide of S. epidermidis, using as controls antibodies induced against teichoic acids and against S. hominus. While type-specific antibodies were highly opsonic, anti-teichoic acid antibodies were not functionally different from the anti-S. hominus antibodies (29).

Similarly, in Kojima et al., the authors assessed the protective effects of antibody to capsular polysaccharide/adhesion against catheter-related bacteremia due to coagulase negative staphylococci and specifically used a strain of S. epidermidis that expresses teichoic acid as a control ((44); see page 436, Materials and Methods, left column, first paragraph; right column, third paragraph). In a later study, Takeda et al. (86), the authors reached a more explicit conclusion against the utility of anti-techoic antibodies:

Immunization protocols designed to elicit antibody to techoic acid but not to PS/A afforded no protection against bacteremia or endocarditis (86).

Thus, the role of antibodies in the protection against infections by Gram positive bacteria, particularly Staphylococci such as S. aureus and S. epidermidis, has not been clear, and there is a need in the, art for polyclonal and monoclonal antibodies to both protect against such bacterial infection and to help elucidate the role of such antibodies against such infection.

Preventing Adherence/Colonization: Various pathogenic species of staphylococci adhere to host factors or artificial surfaces as the first step in their pathogenic process. Blocking of this initial interaction between patient and pathogen, is an effective way to prevent infection. While many staphylococcal factors involved in initial adherence of staphylococci to the patient have been identified, many interactions between patient and pathogen are not yet understood.

Staphylococcal infections are a significant cause of morbidity and mortality, particularly in settings such as hospitals, schools, and infirmaries. Patients particularly at risk include infants, the elderly, the immunocompromised, the immunosuppressed, and those with chronic conditions requiring frequent hospital stays. Further, the advent of multiple drug resistant strains of Staphylococcus aureus increases the concern and need for timely blocking and treatment of such infections. Indeed, the recent World Health Organization report entitled “Overcoming Astionicro Oral Resistance” detailed its concern that increasing levels of drug resistance are threatening to erode the medical advances of the recent decades. Among the issues raised are infections in hospitalized patients. In the United States alone, some 14,000 people are infected and die each year as a result of drug-resistant microbes acquired in hospitals, so called nosocomial infections. Worldwide, as many as 60% of hospital-acquired infections are caused by drug-resistant microbes.

In infections caused by S. aureus, it appears that a principal ecological niche and reservoir for S. aureus is, the human anterior nares. Nasal carriage of staphylococci plays a key role in the epidemiology and pathogenesis of infection (12, 30, 42, 59, 87, 91, 92, 94). In healthy subjects, three patterns of S. aureus nasal carriage can be distinguished over time: approximately 20% of people are persistent carriers, approximately 60% are intermittent carriers, and approximately 20% apparently never carry S. aureus (42).

Nasal carriage of staphylococci is an important risk factor for contracting S. aureus infection. Patients at greatest risk are those undergoing inpatient or outpatient surgery, in the Intensive Care Unit (ICU), on continuous hemodialysis, with HIV infection, with AIDS, burn victims, people with diminished natural immunity from treatments or disease, chronically ill or debilitated patients, geriatric populations, infants with immature immune systems, and people with intravascular devices (12, 30, 35, 42, 43, 52, 59, 92, 94). In one study of ICU patients (20), it was found that on admission 166 of 752 (22%) of patients were S. aureus nasal carriers. The probability of developing a staphylococcal infection was significantly greater (p<0.0001, with a relative risk of 59.6) in these patients than in non-carriers. In 28 out of 30 cases of subsequent staphylococcal infection, identity was found between the S. aureus strain colonizing the nares and the strain isolated from the infection. Even more strikingly, Mest et al. (56) showed that, of 19 patients who were admitted to the ICU with positive nasal cultures for S. aureus, 5 (26%) subsequently developed staphylococcal infections as compared to only 6 S. aureus infections in a group of 465 patients (1.3%) negative for nasal carriage of staphylococci.

Chang et al. (11) studied 84 patients with cirrhosis admitted to a liver transplant unit. Overall, 39 (46%) were nasal carriers of S. aureus and 23% of these patients subsequently developed S. aureus infections as compared to only 4% of the non-carriers. A study of HIV patients (59) showed that 49% (114 of 296) of patients had at least one positive nasal culture for S. aureus. Thirty four percent of 201 patients were considered nasal carriers, with 38% of these being persistent carriers, and 62% intermittent carriers. Twenty-one episodes of S. aureus infection occurred in thirteen of these patients. Molecular strain typing indicated that, for six of seven infected patients, the strain of S. aureus isolated from the infected site was the same as that previously cultured from the nares. The nasal S. aureus carrier patients were significantly more likely to develop S. aureus infection (P=0.04; odds ratio, 3.6; attributable risk, 0.44). This finding led the authors to conclude that nasal carriage is an important risk factor for S. aureus infection in HIV patients (59).

As discussed above, antibiotic resistance continues to be a major problem in staphylococcal infections and the anterior nares is a primary ecological niche for these strains as well. Methicillin resistant S. aureus (“MRSA”) is a well-documented public health problem. In one study performed in a nursing home, 29% of the residents carried S. aureus in the nares and, of those isolates, 31% were MRSA (47). In a separate study of postoperative intra-abdominal infection, it was concluded that MRSA may be a causative pathogen in postoperative intra-abdominal infection and this may be related to nasal colonization (30).

Current technology uses mupirocin ointment to clear staphylococcal nasal colonization. Indeed, antibiotics like mupirocin have been successfully used as intranasal antimicrobial agents in the eradication of nasal carriage of both methicillin sensitive and resistant strains of S. aureus (23, 30, 52, 81, 92). However, mupirocin resistant strains of S. aureus are emerging in many different geographical areas (13, 19, 22, 51).

Until now, staphylococcal factors involved in nasal colonization were unknown. It has now been discovered that WTA, complex surface-exposed polymers covalently linked to the peptidoglycan, are important for S. aureus nasal colonization. This is the first demonstration of a staphylococcal factor that is important for nasal colonization. While WTA has been shown to be important for survival of S. epidermidis and other types of bacteria, defined WTA mutants have never been constructed before. Chemical mutagenesis has resulted in the S. aureus mutant 52A5 (15) which has been proven to lack the enzyme catalyzing the formation of the linkage unit of the WTA (9). However, no gene could be linked to the putative enzyme mediating linkage unit biosynthesis. Soldo and coworkers showed that the Bacillus subtilis tagO homologue was involved in the synthesis of all anionic cell-wall polymers in Bacillus subtilis and catalyzes the formation of the WTA linkage unit in B. subtilis (82, 83). However, tagO was important in B. subtilis and could not be stably deleted. This new work is the first definitive study assigning this role to WTA and defining this molecule as important in the pathogenic process of S. aureus.

Thus, there is also clearly a need in the art for additional interventions for staphylococcal disease.

### SUMMARY OF THE INVENTION

It is a feature of the present invention to provide WTA as a vaccine candidate. Antibodies generated by such a vaccine would serve the dual role of blocking binding of S. aureus to nasal and other epithelium thus preventing the first step in the infectious process.

It is another feature of the present invention to provide a vaccine that generates opsonic antibodies that enhance phagocytosis to protect against S. aureus infection. In one embodiment, the vaccine may comprise WTA. In another embodiment, WTA may be linked to a number of carrier molecules. In yet another embodiment, WTA may be used alone.

It is still another feature of the present invention to provide polyconal or monoclonal antibodies (MAbs) raised against WTA. In one embodiment, the MAbs-may be chimerized. In another embodiment, the MAbs may be humanized. These chimerized or humanized MAbs may, for example, be used to allow use in humans that can be used to interfere with adherence of S. aureus to patient surfaces. MAbs may be used for blocking nasal colonization and other airway colonization such as the first step in S. aureus pneumonia. In another embodiment, these MAbs may be used systemically to treat other S. aureus diseases like, for example, foreign body associated contaminations. The invention also provides methods of treating staphylococcal infections comprising instilling a therapeutically effective amount of a pharmaceutical composition comprising an antibody that specifically binds to WTA or fragments of the antibody to a patient suspected of having a staphylococcal infection.

Yet another feature of the present invention is providing for the identification of human ligands that bind to WTA, as well as the ligands. Such ligands may act as antigens to induce MAbs against these ligands such that the MAbs have same effect of blocking binding of S. aureus to this ligand through WTA.

Still another feature of the present invention provides soluble forms of whole WTA or fragments of WTA produced synthetically or chemically that are used to directly interfere with S. aureus adherence to various surfaces (human and artificial) to block various staphylococcal/patient interactions, e.g., nasal colonization or adherence to other tissue or indwelling devices. This is accomplished by the soluble WTA or fragments thereof competing with the S. aureus bound WTA for their receptor. The invention also provides methods of treating staphylococcal infections comprising instilling a therapeutically effective amount of a pharmaceutical composition comprising soluble forms of whole WTA or fragments of WTA to a patient suspected of having a staphylococcal infection.

Yet a further feature of the present invention is providing for the identification of agents that interfere with the production of WTA in S. aureus, as well as the agents. These agents may also cause the bacteria to lose the capacity to bind to the nasal epithelium and other surfaces in humans and interfere with the infectious process.

Another feature of the present invention is an isolated staphylococcal organism deficient in WTA, wherein the tagO gene has been fully deleted, partially deleted, or rendered non-functional.

The above aspects of the invention are intended only to illustrate the potential for interference of the interaction of S. aureus and the patient through WTA and should not be considered as limiting in the scope of this application. These ideas have use both in humans and veterinary settings.

### BRIEF DESCRIPTION OF THE DRAWINGS

FIG. 1 shows the structure of S. aureus WTA and disruption of WTA production. A. Structure of S. aureus WTA. The N-acetylglucosamine (GlcNAc) phosphate and D-alanine portions are highlighted with gray boxes. MurNAc, N-acetylmuramic acid. B. Location of the S. aureus tagO gene and strategy for its replacement with the ermB cassette. C. Polyacrylamide gel with WTA preparations stained with a combined alcian blue and silver stain procedure. D. The ΔtagO mutant is deficient in WTA. Analysis of the content of phosphate, GlcNAc, and ribitol in WTA preparations from S. aureus Sa113 wild-type (WT), ΔtagO mutant (M) and ΔtagO complemented with plasmid pRBtagO (MC). The mean and SD of at least five independent experiments (phosphate, GlcNAc) or the mean of four counts from one experiment (ribitol) are shown. Ribitol content was determined in WT and M samples only.

FIG. 2 shows the growth characteristics of S. aureus wild-type and ΔtagO. A. Growth curves of S. aureus wild-type (solid circles) and ΔtagO (open circles) in complex (BM) or minimal medium (IMDM) under aeration at 30° C. B. Long-term survival kinetics of wild-type (solid circles) and ΔtagO (open circles) in batch culture.

FIG. 3 shows reduced adherence of ΔtagO and ΔdltA mutants to epithelial cells. A. Primary human bronchial epithelial cells (NHBE). The means and standard deviations (SD) of five independent experiments are shown. B. Primary-human nasal epithelial cells (HNEC). The results of five independent experiments are shown. C. Human airway epithelial cell line A549. The means and SD of at least five independent experiments are shown. The data of three of those five experiments were set forth in priority application 60/430,225. Significant differences vs. wild type samples as calculated with the two-tailed Student's t-test are indicated by one (p<0.05), two (p<0.01), or three (p<0.001) asterisks.

FIG. 4 shows A. Inhibition of S. aureus binding to epithelial cells by wild-type WTA. Confluent layers of A549 cells or HNECs were preincubated with increasing amounts of WTA preparations and adherence of S. aureus strains was analyzed as in a. WTA from wild-type or ΔdltA (0, 125, 250, and 500 μg/ml) or equal volumes of samples from ΔtagO prepared under the same conditions but lacking WTA were used. The means and SD of at least three independent experiments are shown. Significant differences vs. control samples as calculated with the two-tailed Student's t-test are indicated by one (P<0.05), two (P<0.01), or three (P<0.001) asterisks. B. Attachment of WTA-coated latex beads to HNECs and A549 cells. Fluorescent beads were coated with WTA preparations from wild-type (black bars) or ΔdltA (gray bars) or with equal volumes of samples from ΔtagO prepared under the same conditions but lacking WTA (white bars) and hydrophobic bead areas were blocked with BSA. Background adherence of beads incubated without WTA was subtracted. The means and SD of at least five independent experiments are shown. C. IL-8 induction in HNEC by incubation with S. aureus wild-type or ΔtagO. Means and SD of three to four experiments are shown. D. Adherence of S. aureus strains with altered or lacking WTA or deficient in fibronectin-binding proteins were examined for adherence to fibronectin-coated microtiter plates. The ΔfbpA/ΔfbpB 8325-4-derived mutant lacks fbpA and fbpB encoding the two S. aureus Fn-binding proteins.

FIG. 5 shows A. the susceptibilities of S. aureus strains to nasal antimicrobial peptides. Equal numbers of wild-type (black squares), ΔtagO (open circles), and ΔdltA (gray triangles) bacteria were incubated with 100 μg/ml of human defensins hNP1-3, 10 μg/ml cathelicidin LL-37, or 500 μg/ml lactoferrin and viable bacteria were counted after different times of incubation. The means of at least three independent experiments are shown. Significant differences vs. wild-type samples as calculated with the two-tailed, Student's t-test are indicated by one (P<0.05), two (P<0.01), or three (P<0.001) asterisks. B. Lysostaphin-mediated lysis of S. aureus SA113 wild type (solid symbols) and ΔtagO (open symbols). Bacterial suspension with an A₆₀₀ of 1 were incubated at 30° C. for 1 hour with (circles) or without (squares) lysostaphin at a concentration of 1 μg/ml. The values are given as percentages of the initial A₆₀₀.

FIG. 6 shows A. Stability of plasmid pRBtagO in the presence (solid circles) or absence (open circles) of chloramphenicol after repeated cultivation in BM broth. SD is included. B. Inhibition of nasal colonization by preinstillation of cotton rat nares with purified WTA. Ten, six, or twenty animals were used in experiments one, two, or three, respectively. The percentage of animals containing more than ten CFUs of S. aureus per nose are shown. Half of the animals in a particular experiment were either pretreated with PBS only (light gray bars) or with WTA dissolved in PBS (dark gray bars). Further differences in experimental settings are described in detail in the methods section.

### DETAILED DESCRIPTION OF THE INVENTION

Definitions

The term “wall teichoic acid” (WTA), as used herein, includes complex surface-exposed polymers covalently linked to the peptidoglycan in staphylococcal cell walls. WTA also includes soluble whole WTA or fragments thereof. In one embodiment, WTA may be produced synthetically. In another embodiment, WTA may be isolated from staphylococci such as, but not limited to, S. aureus. In another embodiment, WTA may be isolated from a non-staphylococcal organism such as, but not limited to, L. monocytogenes. A WTA preparation is comprised of soluble whole WTA or fragments thereof.

The term “antibody”, as used herein, includes full-length antibodies and portions thereof. A full-length antibody has one pair or, more commonly, two pairs of polypeptide chains, each pair comprising a light and a heavy chain. Each heavy or light chain is divided into two regions, the variable region (which confers antigen recognition and binding) and the constant region (associated with localization and cellular interactions). Thus, a full-length antibody commonly contains two heavy chain constant regions (H_{C} or C_{H}), two heavy chain variable regions (H_{V} or V_{H}), two light chain constant regions (L_{C} or C_{L}), and two light chain variable regions (L_{V} or V_{L}). The light chains or chain, may be-either a lambda or a kappa chain. Thus, in an embodiment of the invention, the antibodies include at least one heavy chain variable region and one light chain variable region, such that the antibody binds an antigen such as WTA.

Another aspect of the invention involves the variable region that comprises alternating complementarity determining regions, or CDRs, and framework regions, or FRs. The CDRs are the sequences within the variable region that generally confer antigen specificity.

The invention also encompasses portions of antibodies that comprise sufficient variable region sequence to confer antigen binding. Portions of antibodies include, but are not limited to Fab, Fab′, F(ab′)₂, Fv, SFv, scFv (single-chain Fv), whether produced by proteolytic cleavage of intact antibodies, such as papain or pepsin cleavage, or by recombinant methods, in which the cDNAs for the intact heavy and light chains are manipulated to produce fragments of the heavy and light chains, either separately, or as part of the same polypeptide.

Antibodies within the scope of the invention include sequences corresponding to human antibodies, animal antibodies, and combinations thereof. In one embodiment, antibody preparations comprise polyclonal antibodies. In another embodiment, antibody preparations comprise monoclonal antibodies. In further embodiments, antibody preparations comprise chimeric antibodies. In another embodiment, antibody preparations comprise humanized antibodies. The term “chimeric antibody,” as used herein, includes antibodies, derived from monoclonal antibodies, that have variable regions derived from an animal antibody, such as a rat or mouse antibody, fused to another molecule, for example, the constant domains derived from a human antibody. One type of chimeric antibodies, “humanized antibodies”, have had the variable regions-altered (through mutagenesis or CDR grafting) to match (as much as possible) the known sequence of human variable regions. CDR grafting involves grafting the CDRs from an antibody with desired specificity onto the FRs of a human antibody, thereby replacing much of the non-human sequence with human sequence. Humanized antibodies, therefore, more closely match (in amino acid sequence) the sequence of known human antibodies. By humanizing mouse monoclonal antibodies, the severity of the human anti-mouse antibody, or HAMA, response is diminished. The invention-further includes fully human antibodies which would avoid, as much a possible, the HAMA response.

Modified antibodies include, for example, the proteins or peptides encoded by truncated or modified antibody-encoding genes. Such proteins or peptides may function similarly to the antibodies of the invention. Other modifications, such as the addition of other sequences that may enhance the effector function, which includes the ability to block or alleviate nasal colonization by staphylococci, are also within the present invention. Such modifications include, for example, the addition of amino acids to the antibody's amino acid sequence, deletion of amino acids in the antibody's amino acid sequence, substitution of one or more amino acids in the antibody amino acid sequence with alternate amino acids, isotype switching, and class switching.

In certain embodiments, an antibody may be modified in its Fc region to prevent binding to bacterial proteins. The Fc region normally provides binding sites for neutrophils, macrophages, other accessory cells, complement components, and, receptors of the immune system. As the antibodies bind to bacteria and opsonize them, accessory cells recognize the coated bacteria and respond to infection. When a bacterial protein binds to the Fc region near the places where accessory cells bind, the normal function of these cells is inhibited. For example, Protein A, a bacterial protein found in the cell membrane of S. aureus, binds to the Fc region of IgG near accessory cell binding sites. In doing so, Protein A inhibits the function of these accessory cells, thus interfering with clearance of the bacterium. To circumvent this interference with the antibacterial immune response, the Fc portion of the antibody of the invention may be modified to prevent nonspecific binding of Protein A while retaining binding to accessory cells (15).

In light of these various forms, the antibodies of the invention include clones of full length antibodies, antibody portions, chimeric antibodies, humanized antibodies, fully human antibodies, and modified antibodies. Collectively, these will be referred to as “MAbs” or monoclonal antibodies unless otherwise indicated.

The term “epitope”, as used herein, refers to a region, or regions, of WTA that is bound by an antibody to WTA. The regions that are bound may or may not represent a contiguous portion of the molecule.

The term “antigen”, as used herein, refers to a polypeptide sequence, a non-proteinaceous molecule, or any molecule that can be recognized by the immune system. An antigen may be a full-sized staphylococcal protein or molecule, or a fragment thereof, wherein the fragment is produced from a recombinant cDNA encoding less than the full-length protein; derived from the full-sized molecule or protein; produced synthetically; or isolated from an organism such as, but not limited to, staphylococci. Fragments may also be produced via enzymatic processing, such as proteolysis. An antigen may also be a polypeptide sequence that encompasses an epitope of a staphylococcal protein, wherein the epitope may not be contiguous with the linear polypeptide sequence of the protein. The DNA sequence encoding an antigen may be identified, isolated, cloned, and transferred to a prokaryotic or eukaryotic cell for expression by procedures well-known in the art (64).

An antigen, or epitope thereof, may be 100% identical to a region of the staphylococcal molecule or protein amino acid sequence, or it may be at least 95% identical, or at least 90% identical, or at least 85% identical. An antigen may also have less than 95%, 90% or 85% identity with the staphylococcal molecule or protein amino acid sequence, provided that it still be able to elicit antibodies the bind to a native staphylococcal molecule or protein. The percent identity of a peptide antigen can be determined, for example, by comparing the sequence of the target antigen or epitope to the analogous portion of staphylococcal sequence using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443,1970), as revised by Smith and Waterman (Adv. Appl. Math 2:482, 1981), and is applicable to determining the percent identity of protein or nucleotide sequences referenced herein. The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

Alternatively, for simple comparisons over short regions up to 10 or 20 units, or regions of relatively high homology, for example between antibody sequences, the percent identity over a defined region of peptide or nucleotide sequence may by determined by dividing the number of matching amino acids or nucleotides by the total length of the aligned sequences, multiplied by 100%. Where an insertion or gap of one, two, or three amino acids occurs in a MAb chain, for example in or abutting a CDR, the insertion or gap is counted as single amino acid mismatch.

Antigens include, but are not limited to surface antigens, virulence antigens, and adherence antigens. Surface antigens are antigens that are accessible to an antibody when the antigen is in the configuration of the whole intact bacterium, i.e., the antigen is not inside the cell cytoplasm. Virulence antigens are antigens that are involved in the pathogenic process, causing disease in a patient. Virulence antigens include, for example, lipoteichoic acid (LTA), peptidoglycan, toxins, fimbria, flagella, and adherence antigens. Adherence antigens such as WTA mediate the ability of a staphylococcal bacterium to adhere to an epithelial surface, such as the epithelial surface of the anterior nares. An antigen may be a non-proteinaceous component of staphylococci such as a carbohydrate or lipid. For example, peptidoglycan, LTA, and WTA are non-proteinaceous antigens found in the cell wall of staphylococci. Antigens may comprise or include fragments of non-proteinaceous molecules as long as they elicit an immune response.

As used herein, antigens include molecules that can elicit an antibody response to WTA. An antigen may be WTA itself, or a fragment or portion thereof. An antigen may also be an unrelated molecule, which, through some structural similarity, is able to elicit antibodies that bind to WTA. Binding to WTA may thus be assessed by binding to such peptide epitope mimics, as described, for example, in U.S. Ser. No. 09/893,615, incorporated herein by reference. In certain embodiments of the invention, an antigen elicits antibodies that bind to WTA on the surface of bacteria. As specifically used herein, an antigen is any molecule that can specifically bind to an antibody, including antibodies specific for WTA.

An antibody is said to specifically bind to an antigen, epitope-, or protein, if the antibody gives a signal by an assay such as an ELISA assay that is at least two fold, at least three fold, at least five fold, or at least ten fold greater than the background signal, i.e., at least two fold, at least three fold, at least five fold, or at least ten fold greater than the signal ascribed to non-specific binding. An antibody is said to specifically bind to a bacterium if the antibody gives a signal by methanol-fixed bacteria ELISA or live bacteria ELISA, or other assay, that is at least 1.5 fold, 2 fold, or 3 fold greater than the background signal.

“Enhanced phagocytosis”, as used herein, means an increase in phagocytosis over a background level as assayed by the methods in this application, or another comparable assay. The level deemed valuable may well vary depending on the specific circumstances of the infection, including the type of bacteria and the severity of the infection. For example, for enhanced phagocytic activity, in one embodiment, an enhanced response is equal to or greater than 75% over background phagocytosis. In another embodiment, an enhanced response is equal to or greater than 80% or 85% over background phagocytosis. In another embodiment, an enhanced response is equal to or greater than 90% or 95% over background phagocytosis. Enhanced phagocytosis may also be equal to or greater than 50%, 55%, 60%, 65%, or 70% over background phagocytosis. In another embodiment, enhanced phagocytosis comprises a statistically significant increase in phagocytic activity as compared to background phagocytosis or phagocytosis with a non-specific or non-opsonic control antibody. An antibody has “opsonic activity” if it can bind to an antigen to promote attachment of the antigen to the phagocyte and thereby enhance phagocytosis. As used herein, opsonic activity may also be assessed by assays that measure neutrophil mediated opsonophagocytotic bactericidal activity.

The specific determination or identification of a “statistically significant” result will depend on the exact statistical test used. One of ordinary skill in the art can readily recognize a statistically significant result in the context of any statistical test employed, as determined by the parameters of the test itself. Examples of these well-known statistical tests include, but are not limited to, X² Test (Chi-Squared Test), Students t Test, F Test, M test, Fisher Exact Text, Binomial Exact Test, Poisson Exact Test, one way or two way repeated measures analysis of variance, and calculation of correlation efficient (Pearson and Spearman).

The antibody preparations of the invention and the WTA preparations of the invention are useful for the treatment of systemic and local staphylococcal infections in a patient. Local infections are found in specific areas of a patient's body, such as, but not limited to, the nose. As used herein, “patient” includes humans and non-human mammals which are hosts for bacterial infections, including, but not limited to, staphylococcal infections. As used herein, “treatment” encompasses any reduction, amelioration, or “alleviation” of existing infection as well as “blocking” or prophylaxis against future infection. In this respect, treatment with an antibody preparation of the invention or a WTA preparation of the invention is said to “alleviate” staphylococcal colonization if it is able to decrease the number of colonies in the nares of a mammal when the MAb or WTA preparation is administered before, concurrently with, or after exposure to staphylococci, whether that exposure results from the intentional instillation of staphylococcus or from general exposure. For instance, in the nasal colonization animal model described below, an antibody preparation or WTA preparation is considered to alleviate colonization if the extent of colonization, or the number of bacterial colonies that can be grown from a sample of nasal tissue, is decreased after administering the antibody preparation or WTA preparation. An antibody preparation or WTA preparation alleviates colonization in the nasal colonization assays described herein when it reduces the number of colonies by at least 50%, at least 60%, at least 75%, at least 80%, or at least 90%. 100% alleviation may also be referred to as eradication.

An antibody preparation or WTA preparation is said to “block” staphylococcal colonization if it is able to prevent the nasal colonization of a human or non-human mammal when it is administered prior to, or concurrently with, exposure to staphylococci, whether by intentional instillation or otherwise into the nares. An antibody preparation or WTA preparation blocks colonization, as in the nasal colonization assay described herein, if no staphylococcal colonies can be grown from a sample of nasal tissue taken from a mammal treated with the MAb of the invention for an extended period such as 12 hours or longer or 24 hours or longer compared to control mammals. An antibody preparation or WTA preparation also blocks colonization in the nasal colonization assay described herein if it causes a reduction in the number of animals that are colonized relative to control animals. For instance, an antibody preparation or WTA preparation is considered to block colonization if the number of animals that are colonized after administering the material and the Gram-positive bacteria is reduced by at least 25%, at least 50%, and at least 75%, relative to control animals or if no colonies can be grown from a sample taken from a treated individual for an extended period such as 12 hours or 24 hours or longer.

In a clinical setting, the presence or absence of nasal colonization in a patient is determined by culturing nasal swabs on an appropriate bacterial medium. These cultures are scored for the presence or-absence of staphylococcal colonies. In this type of qualitative assay system, it may be difficult to distinguish between blocking and alleviation of staphylococcal colonization. Thus, for the purposes of qualitative assays, such as nasal swabs, an antibody preparation or WTA preparation “blocks” colonization if it prevents future colonization in human patients who show no signs of prior colonization for an extended period of 12 or 24 hours or longer. An antibody preparation or WTA preparation “alleviates” colonization if it causes a discernable decrease in the number of positive cultures taken from a human patient who is already positive for staphylococci before the antibody preparations or the WTA preparations of the invention are administered.

A “vaccine” as used herein includes pharmaceutical compositions comprising antibodies or antigens. In one embodiment, vaccines comprise a preparation of soluble whole WTA or a fragment thereof. In another embodiment, vaccines comprise a preparation of antibodies that specifically bind to WTA. In another embodiment, vaccines comprising a preparation of antibodies that specifically bind to WTA are used in passive immunotherapy. In yet another embodiment, vaccines also comprise pharmaceutically acceptable carriers, as further discussed below.

A vaccine is considered to confer a protective immune response if it stimulates the production of opsonic antibodies with enhanced phagocytic activity to Gram-positive bacteria. Production of opsonic antibodies may be measured by the presence of such antibodies in the serum of a test subject that has been administered the vaccine, relative to a control that has not received the vaccine. The presence of opsonic antibodies in the serum may be measured by, for example, an opsonophagocytic bactericidal assay as described in U.S. Pat. No. 6,610,293. Alternatively, such an assay may be carried out by using neutrophils isolated from adult venous blood by sedimentation using PMN Separation Medium (Robbins Scientific catalog no.1068-00-0). Antibody, serum, or other immunoglobulin source, is added at various dilutions to replicate wells of a round-bottom microtiter plate. Neutrophils (approximately 2×10⁶ cells per well) are then added to each well, followed immediately by approximately 3×10 mid-log phase bacteria in 10 μl Tryptic Soy Broth or other suitable bacterial growth medium. Immunoglobulin-depleted human serum is added as a source of active complement. (Immunoglobulins are removed from human serum complement by preincubating the serum with Protein G-agarose and Protein L-agarose before use in the assay. This depletion of immunoglobulins minimizes the concentrations of anti-staphylococcal antibodies in the complement, thereby reducing bacterial killing caused by inherent antibodies in the complement solution.)

The plates are incubated at 37° C. with constant vigorous shaking. Aliquots are taken from each well at zero time, when the sample antibody is first added, and after 2 hours of incubation. To determine the number of viable bacteria in each aliquot harvested from each sample well, each aliquot is diluted 20-fold in a solution of 0.1% BSA in water (to lyse the PMNs), mixed vigorously by rapid pipetting, and cultured on blood agar plates (Remel, cat. no. 01-202, or equivalent) overnight at 37° C. The opsonic activity is measured by comparing the number of bacterial colonies observed from the sample taken at two hours with the number of bacterial colonies observed from the sample taken at time zero. Colonies are enumerated using an IPI Minicount Colony Counter.

A vaccine enhances immunity when the test serum generated by administering the vaccine results in the killing of at least 50% more bacteria, 75% more bacteria, and at least 100% more bacteria, relative to the control serum of a non-vaccinated patient.

### DETAILED DESCRIPTION OF THE INVENTION

The present invention provides antibodies, including monoclonal antibodies, and chimeric, humanized and fully human antibodies, fragments, derivatives, and regions thereof, which bind to WTA of Gram positive staphylococci. In one embodiment, the antibodies of the invention bind to the patient ligand that staphylococcal WTA binds to. These anti-ligand antibodies may, for example, inhibit the binding of staphylococci to patient surfaces by inhibiting the interaction of WTA with its ligand. Gram positive bacteria, unlike Gram negative bacteria, take up the Gram stain as a result of a difference in the structure of the cell wall. The cell walls of Gram negative bacteria are made up of a unique outer membrane of two opposing phospholipid-protein leaflets, with an ordinary phospholipid in the inner leaflet but the extremely toxic lipopolysaccharide in the outer leaflet. The cell walls of Gram positive bacteria seem much simpler in comparison, containing two major components, peptidoglycan and teichoic acids plus additional carbohydrates and proteins depending on the species. Though the structure of WTA differs between different staphylococcal species, antibodies raised against S. aureus WTA may recognize some common WTA modifications such as D-Alanine esters or GlcNAc modification and cross react with WTA from other staphylococcal species. Moreover anti-WTA antibodies may also specifically bind non-staphylococccal species. For example, Listeria monocytogenes has the same WTA structure as S. aureus. Thus, antibodies that specifically bind S. aureus WTA may also specifically bind L. monocytogenes.

Among the Gram positive staphylococci against which the antibodies of the invention are directed are S. aureus (a coagulase positive bacteria) and S. epidermidis (a coagulase negative bacteria).

In one embodiment, the invention relates to antibodies that bind to the WTA of Gram positive bacteria. In another embodiment, these antibodies enhance the phagocytosis of such bacteria. These anti-WTA antibodies include, but are not limited to, polyclonal antibodies, MAbs, and other MAbs antibodies including, chimeric, humanized, fully human antibodies, antibody fragments, and modified antibodies. Chimeric or other monoclonal antibodies are advantageous in that they avoid the development of anti-murine antibodies. In at least one study, patients administered murine anti-TNF (tumor necrosis factor) monoclonal antibodies developed anti-murine antibody responses to the administered antibody (28). This type of immune response to the treatment regimen, commonly referred to as the human anti-mouse antibody response, or the HAMA response, decreases the effectiveness of the treatment and may even render the treatment completely ineffective. Humanized or chimeric human/non-human monoclonal antibodies have been shown to significantly decrease the HAMA response and to increase the therapeutic effectiveness (49).

Thus, in one aspect of the invention, a chimeric heavy chain can comprise the antigen binding region of the heavy chain variable region of the anti-WTA antibody of the invention linked to at least a portion of a human heavy chain IgG, IgA, IgM, or IgD constant region. This humanized or chimeric heavy chain may be combined with a chimeric light chain that comprises the antigen binding region of the light chain variable region of the anti-WTA antibody linked to at least a portion of the human light chain kappa or lambda constant region. Exemplary embodiments include, but are not limited to, an antibody having a mouse heavy chain variable region fused to a human IgG, constant region, and a mouse light chain variable region fused to a human kappa light chain constant region.

The chimeric antibodies and other MAbs of the invention may be monovalent, divalent, or polyvalent immunoglobulins. For example, a monovalent chimeric antibody is a dimer (HL) formed by a chimeric H chain associated through disulfide bridges with a chimeric L chain, as noted above. A divalent chimeric antibody is a tetramer (H₂ L₂) formed by two HL dimers associated through at least one disulfide bridge. A polyvalent or multivalent chimeric antibody may be based on an aggregation of chains, with or without a carrier or scaffold.

The MAbs of the invention include antibodies that contain heavy and light chain variable regions derived from two different antibodies. In one embodiment, the heavy and light chain variable regions are derived from two antibodies that bind to the same molecule, e.g. WTA.

In addition to the antibodies, the present invention also encompasses the DNA sequences of the genes coding for the antibodies as well as the polypeptides encoded by the DNA.

The invention includes peptide sequences for, and DNA sequences encoding, full-length antibodies and portions thereof, as well as CDRs and FRs relating to these MAbs. The invention further includes DNA and peptide sequences that are homologous to these sequences. In one embodiment, these homologous DNAs and peptide sequences are about 70% identical, although other embodiments include sequences that about 75%, 80%, 85%, 88%, 90%, and 95% or more identical. As indicated above, determining levels of identity for both the DNA and peptide sequence is well within the routine skill of those in the art.

In another embodiment, the invention contemplates production systems for MAbs, light chains, heavy chains, and portions thereof, comprising 1) a cell (including-bacteria, yeast, microorganisms, eukaryotic cell lines, transgenic plant or animal) in connection with 2) at least one recombinant nucleic acid capable of directing the expression of any of the MAbs or related polypeptides of the invention.

The DNA sequences of the invention can be identified, isolated, cloned, and transferred to a prokaryotic or eukaryotic cell for expression by procedures well-known in the art. Such procedures are generally described in Molecular Cloning: A Laboratory Manual, as well as Current Protocols in Molecular Biology (5, 76), which are incorporated by reference. Guidance relating more specifically to the manipulation of sequences of the invention may be found in Antibody Engineering, and Antibodies: A Laboratory Manual (8, 39), both of which are incorporated by reference in their entirety. In certain embodiments, a CDR can be grafted onto any human antibody framework region using techniques standard in the art, in such a manner that the CDR maintains the same binding specificity as in the intact antibody. As noted as above, an antibody that has its CDRs grafted onto a human framework region is said to be “humanized”. Humanized, and fully human antibodies generally also include human constant regions, thus maximizing the percentage of the antibody that is human-derived, and potentially minimizing the HAMA response.

In addition, the DNA and peptide sequences of the antibodies of the invention, including both monoclonal and chimeric antibodies, humanized and fully human antibodies, may form the basis of antibody “derivatives,” which include, for example, the proteins or peptides encoded by truncated or modified genes. Such proteins or peptides may function similarly to the antibodies of the invention. Other modifications, such as the addition of other sequences that may enhance the effector function, which includes phagocytosis and/or killing of the bacteria, are also within the present invention.

The present invention also discloses a vaccine comprising antibodies specific for WTA, whether monoclonal or chimeric, humanized, or fully human, together with a pharmaceutically acceptable carrier. In one embodiment, a vaccine comprising antibodies specific for WTA may be used to block the adherence of staphylococci to patient surfaces, which include but are not limited to the nares, the skin, and airway epithelia such as in the lung. In one embodiment, the antibody vaccine may be used to block or treat bacterial infections in patients susceptible to lung infections, such as, but not limited to, patients with cystic fibrosis. In another embodiment, a vaccine comprising antibodies specific for WTA may be used to systemically treat a patient to block or alleviate staphylococcal contaminations associated with foreign bodies, which include but are not limited to catheters and prosthetics (i.e., an artificial knee or hip joint).

The vaccines of the invention may alternatively comprise the isolated WTA or portions thereof, together with a pharmaceutically acceptable carrier. In one embodiment, a WTA vaccine may be used to induce the production of antibodies that bind to WTA. The invention also includes medicinal compositions comprising WTA. In one embodiment, a medicinal composition may interfere with the interaction of staphylococci with surfaces by coating the surface with WTA. Surfaces include, but are not limited to, patient surfaces and artificial surfaces such as those found in prosthetics or catheters. The vaccine WTA competes with WTA in the staphylococcal cell wall for binding to these surfaces:

Pharmaceutically acceptable carriers can be sterile liquids, such as water, oils, including petroleum oil, animal oil, vegetable oil, peanut oil, soybean oil, mineral oil, sesame oil, and the like. Saline solutions, aqueous dextrose, and glycerol solutions can also be employed as liquid carriers, particularly for injectable solutions. Suitable pharmaceutical carriers are described in Remington's Pharmaceutical Sciences, 18th Edition (36), which is herein incorporated by reference.

Additionally, the invention may be practiced with various delivery vehicles and/or carriers. Such vehicles may increase the half-life of the MAbs or WTA in storage and upon administration including, but not limited to, application to skin, wounds, eyes, lungs, or mucus membranes of the nasal or gastrointestinal tract, or upon inhalation or instillation into the nares. These carriers comprise natural polymers, semi-synthetic polymers, synthetic polymers, lipososmes, and semi-solid dosage forms (55, 57, 74, 78, 84, 85). Natural polymers include, for example, proteins and polysaccharides. Semi-synthetic polymers are modified natural polymers such as chitosan, which is the deacetylated form of the natural polysaccharide, chitin. Synthetic polymers include, for example, polyphosphoesters, polyethylene glycol, poly (lactic acid), polystyrene sulfonate, and poly (lactide coglycolide). Semi-solid dosage forms include, for example, dendrimers, creams, ointments, gels, and lotions. These carriers can also be used to microencapsulate the MAbs or be covalently linked to the MAbs.

The present invention provides methods for treating a patient infected with, or suspected of being infected with, a Gram-positive bacteria such as a staphylococcal organism. In one embodiment, the method comprises administering a therapeutically effective amount of a vaccine comprising the anti-WTA immunoglobulin (whether monoclonal, chimeric, humanized, or fully human, including fragments, regions, and derivatives thereof) and a pharmaceutically acceptable carrier. In another embodiment, the method comprises administering a therapeutically effective amount of a vaccine comprising WTA or a fragment thereof and a pharmaceutically acceptable carrier. Representative patients include any mammal subject to S. aureus or other staphylococcal or Gram-positive infection or carriage, including humans and non-human animals such as mice, rats, rabbits, dogs, cats, pigs, sheep, goats, horses, primates, ruminants including beef and milk cattle, buffalo, camels, as well as fur-bearing animals, herd animals, laboratory, zoo, and farm animals, kenneled and stabled animals, domestic pets, and veterinary animals.

A therapeutically effective amount is an amount reasonably believed to provide some measure of relief, assistance, prophylaxis, or preventative effect in the treatment of the infection. A therapeutically effective amount may be an amount believed to be sufficient to block a bacterial infection. Similarly, a therapeutically effective amount may be an amount believed to be sufficient to alleviate a bacterial infection. Such therapy as above or as described below may be primary or supplemental to additional treatment, such as antibiotic therapy, for a staphylococcal infection, an infection caused by a different agent, or an unrelated disease. Indeed, combination therapy with other antibodies is expressly contemplated within the invention.

The antibody preparations and WTA preparations of the invention may be administered in conjunction with other antibiotic anti-staphylococcal drugs including antibiotics like mupirocin and bacitracin; anti-staphylococcal agents like lysostaphin, lysozyme, mutanolysin, and cellozyl muramidase; anti-bacterial peptides like nisin; and other lantibiotics, or any other lanthione-containing molecule, such as nisin or subtilin.

A further embodiment of the present invention is a method of preventing such infections, comprising administering a prophylactically effective amount of a vaccine comprising the anti-WTA antibody (whether monoclonal, chimeric, humanized, or fully human) and a pharmaceutically acceptable carrier. In another embodiment, the present invention is a method of preventing such infections, comprising administering a prophylactically effective amount of a vaccine comprising WTA or a fragment thereof and a pharmaceutically acceptable carrier

A prophylactically effective amount is an amount reasonably believed to provide some measure of prevention of infection by Gram positive bacteria. Such therapy as above or as described below may be primary or supplemental to additional treatment, such as antibiotic therapy, for a staphylococcal infection, an infection caused by a different agent, or an unrelated disease. Indeed, combination therapy with other antibodies is expressly contemplated within the invention.

The vaccines of the invention may be administered by intravenous, intraperitoneal, intracorporeal injection, intra-articular, intraventricular, intrathecal, intramuscular or subcutaneous injection, or intranasally, dermally, intradermally, intravaginally, orally, or by any other effective method of administration. The composition-may also be given locally, such as by injection to the particular area infected, either intramuscularly or subcutaneously. Administration can comprise administering the vaccine by swabbing, immersing, soaking, or wiping directly to a patient. The treatment can also be applied to objects to be placed within a patient, such as indwelling catheters, cardiac valves, cerebrospinal fluid shunts, joint prostheses, other implants into the body, or any other objects, instruments, or appliances at risk of becoming infected with a Gram positive bacteria, or at risk of introducing such an infection into a patient.

As a particularly valuable corollary of treatment with the compositions of the invention (pharmaceutical compositions comprising anti-WTA antibodies, whether, monoclonal, chimeric, humanized or fully human) may be the reduction in cytokine release that results from the introduction of the WTA of a Gram positive bacteria (54). WTA may induce cytokines. Accordingly, the compositions of the invention may enhance protection at three levels: (1) by binding to WTA on the bacteria and thereby blocking the initial binding to epithelial cells and preventing subsequent invasion of the bacteria; (2) by binding to WTA on bacteria and thereby enhancing opsonization of the bacteria and clearance of the bacteria from tissues and/or blood; and/or (3) by binding to WTA and partially or fully blocking cytokine release and modulating the inflammatory responses to prevent shock and tissue destruction.

Finally, the invention provides for staphylococcal organisms that are deficient in WTA. In one embodiment, the staphylococcal organism is S. aureus. The term “deficient in WTA” as used herein means that the staphylococcal organism does not contain WTA in its cell wall. A staphylococcal organism deficient in WTA may be “constructed” using techniques, such as recombinant DNA techniques, that are known to those in the art. For example, staphylococcal organism deficient in WTA may be constructed by inactivating a staphylococcal gene that produced a biological product that is involved in synthesizing WTA or incorporating WTA into the bacterial cell wall. Such genes include, but are not limited to, the tagO gene. A gene is “inactivated” when the biological product the gene encodes is absent from the cell or when the biological product no longer performs its normal function in the cell. A gene may be inactivated by several methods, including but not limited to, deleting either the entire gene or a portion of the gene from a staphylococcal organism's genome; changing the gene's nucleotide sequence at one or more nucleotide positions; or by adding additional nucleotide sequences to the gene's nucleotide sequence (i.e., to disrupt the gene). Staphylococcal organisms deficient in WTA may be used, for example, to study the effect of WTA on the patient's immune response to staphylococci by comparing immune responses to the WTA deficient mutant to immune responses to the wild-type strain from which the WTA deficient mutant was generated.

Having generally described the invention, it is clear that the invention overcomes some of the potentially serious problems described in the Background section regarding the development of antibiotic resistant Gram positive bacteria. As set forth above, Staphylococci and Streptococci (such as S. faecalis) have become increasingly resistant to antibiotics and, with the recent spread of drug resistant strains, antibiotic therapy may become totally ineffective.

Particular aspects of the invention are now presented in the form of the following Materials and Methods, as well as the specific Examples. Of course, these are included only for purposes of illustration and are not intended to be limiting of the present invention.

### Materials and Methods

Bacterial strains and growth conditions: S. aureus SA113 and 8325-4 are laboratory strains frequently used in experimental infection studies (61). The SA113 dltA mutant and plasmid pRBdlt1 used for complementation of the mutant have recently been described in detail (68). A 8325-4-derived mutant that lacks fbpA and fbpB encoding the two S. aureus Fn-binding proteins was kindly provided by Tim J. Foster (Dublin, Ireland) (37) All bacterial strains were grown in LB broth (1% tryptone, 0.5% yeast extract, 0.5% NaCl) or BM broth (LB supplemented with 0.1% K₂HPO₄ and 0.1% glucose) at 37° C. unless otherwise noted. In order to compare the generation times of wild-type and mutant bacteria, complex medium (BM) or synthetic minimal medium (IMDM without phenol red, Gibco-BRL, Carlsbad, Calif.) was inoculated with 1/100 volumes of overnight cultures, 200-μl samples were added to the wells of 96-well microtiter plates and vigorously shaken at 30° C. or 37° C. Bacterial densities were determined in a microplate reader at several time points and generation times were calculated for log-phase growth.

Generation and complementation of a tagO mutant: Gene SA0702 from the S. aureus genome (27) exhibits 62% similarity to the B. subtilis tagO. In order to inactivate the corresponding gene, DNA fragments consisting of 1001 bp upstream and 1002 bp downstream of SA0702 were amplified by PCR from S. aureus SA113 (ATCC 35556) DNA and cloned using KpnI/SalI (upstream) and EcORI/XbaI (downstream) restriction sites together with the SalI/EcORI-digested ermB gene from Tn551 into the KpnI/XbaI-digested temperature-sensitive shuttle plasmid pBT2. After cloning in E. coli DH5α the sequence of the PCR products was confirmed. The resulting plasmid pBTΔtagO was transformed into S. aureus SA113 to achieve integration of the ermB gene into the genome by homologous recombination. Mutants were enriched by cultivation at 42° C. in the presence of 2.5 μg/ml erythromycin. The proper integration of ermB, which is considerably larger than tagO, was confirmed by PCR analysis. A 1055-bp fragment encoding the entire TagO was deleted. E. coli- and Staphylococcus-specific plasmid vectors and molecular methods have been described previously (4, 5, 10, 61, 67). The procedures for cloning, homologous recombination, and verification of the mutant were performed as essentially described recently (10, 68, 69).

Plasmid pRBtagO was constructed by cloning a 1720-bp PCR fragment bearing the tagO gene together with the putative promoter region (460-bp non-coding upstream DNA). The PCR primers had been modified to introduce Asp7181 (upstream) and HindIII (downstream) restriction sites. Primer sequences were as follows:

| | | |
|---|---|---|
| TO-PCR1 (Asp7181) | 5′ GGATAAGGGATAGGGTACCCAGATATAAATAATGATACG 3′ | (SEQ ID NO. 1) |
| TO-PCR2 (HindIII) | 5′ GAAGAAACTCCAAAGCTTTTATTCGATATACCAAACT 3′ | (SEQ ID NO. 2) |

These PCR primers permitted ligation of the fragment into the shuttle vector pRB473 (67) digested with the same restriction sites. pRBtagO was constructed in E. coli DH5α and transformed into S. aureus SA113 by electroporation (4). In order to analyze the stability of plasmid pBRtagO, bacteria were cultivated in consecutive cultures lacking antibiotic, which were inoculated 1:100 every 24 hours. Viable bacteria were counted by plating diluted bacterial suspensions on agar with or without chloramphenicol.

Isolation and characterization of WTA: Staphylococcal teichoic acids were isolated and analyzed as described recently (68). Briefly, bacteria were grown overnight in BM broth containing 0.3% glucose, re-suspended in sodium acetate buffer (20 mM, pH 4.6) and disrupted using glass beads. Cell walls were prepared by extraction of crude cell lysates with 2% SDS followed by extensive washing with sodium acetate buffer. Cell wall teichoic acids were released by treatment of the cell walls with 5% trichloroacetic acid. The amounts of phosphorus and hexosamines in teichoic acid samples were determined by calorimetric assays as described elsewhere (18, 68, 80). The ribitol content was determined by gas chromatography. 100 μl of WTA preparations were heated with 100 μl 6N HCl at 110° C. for 23 h; under these conditions ribitol is converted completely to anhydroribitol. 100 μl methanol and 10 μl tert-butanol were added and the sample dried in a vacuum centrifuge. Ribitol was then derivatized with 50 μl bis(trimethylsilyl)trifluoroacetamide/acetonitrile (1:1) at 110° C. for 2 h. The samples were diluted with 100 μl methylene chloride containing 20 μg n-tetracosane (internal standard) and analyzed on a DB 5 fused silica capillary (30 m×0.25 mm; df=0.1 μm; J+W Scientific, Folsom, Calif.) using a HP 5890 gas chromatograph with a flame ionization detector and H₂ as carrier gas (Hewlett Packard, Palo Alto, Calif.). The response factor of ribitol relative to n-tetradecane was determined under identical conditions of sample preparation.

For some assays WTA was further purified by ethanol precipitation as described previously (54). In brief, WTA was allowed to precipitate for 15 hours after addition of 1/10 volume of 3 M sodium acetate (pH 5.1.) and 3 volumes of 95% ice-cold ethanol. After centrifugation at 8000×g for 20 min at 4° C. WTA was washed three times with 80% ethanol and lyophilized.

In order to separate WTA by polyacrylamide gel electrophoresis (PAGE) precipitated WTA was dissolved in a sample buffer (50 mM Tris/HCl, pH 6.8, 10% glycerol, 2.5% bromophenol blue), applied to tris/tricine gels (77) containing 18% acrylamide and lacking SDS, and visualized by a combined alcian blue and silver stain, essentially as described elsewhere (70).

Phages 3A, φ11, and 85 were propagated in S. aureus SA113 wild-type according to standard procedures (34, 61) and their activity was studied by dropping phage suspensions on lawns of S. aureus strains as described recently (18). Bacterial growth was studied in BM broth (1% tryptone, 0.5% yeast extract, 0.5% NaCl, 0.1% K₂HPO₄, and 0.1% glucose) or IMDM (Gibco-BRL) under vigorous aeration after inoculation of the medium with 1/100 of an overnight culture. Survival rates in the stationary phase were investigated in the same way using BM broth.

Cotton rat model of nasal colonization: The cotton rat nasal colonization model has recently been described in detail (45). Briefly, S. aureus was grown overnight on Columbia agar (BD, Sparks, Md.) supplemented with 2% NaCl (Sigma, St. Louis, Mo.) to induce capsule formation. Plate-grown bacteria were washed by suspension in phosphate buffered saline (PBS, BioWhitakker) so that the percent transmission of the suspension was less than 10%. A volume of suspended bacteria equivalent to 1 ml per animals to be instilled was pelleted by centrifugation and then resuspended in 10 μl PBS per animal to be instilled containing no antibiotics or, in one experiment, 2.5 μg/ml erythromycin, 300 μg/ml spectinomycin, or 10 μg/ml chloramphenicol for ΔtagO, ΔdltA, or complemented tagO respectively. Six week-old female cotton rats (Sigmadon hispidis) were anesthetized with a combination of Rompun, acepromazine maleate, and Ketamine (2.5 mg/kg, 2.5 mg/kg and 25 mg/kg respectively). A 10 μlu aliquot of resuspended S. aureus (109 CFUs) was intranasally instilled in a drop-wise fashion distributed equally in each nostril of the anesthetized animal.

Seven days after nasal instillation of the bacteria, animals were sacrificed, the nose area was cleansed thoroughly with 70% ethanol to eliminate surface colonization by S. aureus, and the noses were surgically removed. The nostrils were bisected with scissors and then the excised nose was placed in 500 μl of PBS containing 0.5% Tween-20 (Sigma). The nose was vortexed vigorously three times for 20 seconds each to release colonizing bacteria and 50-100 μl of supernatant was plated on blood agar (Remel, Lenexa, KN) or tryptic soy agar (TSA, BD) supplemented with 7.5% NaCl. Antibiotics (spectinomycin 300 μg/ml, erythromycin 2.5 μg/ml or chloramphenicol 10 μg/ml, Sigma) or lysostaphin (1 μg/ml) were added to the TSA in some experiments to aid in isolation of the strain of S. aureus used in the particular study. TSA plates supplemented with NaCl were incubated for 48 hours at 37° C. to allow S. aureus growth.

To differentiate nasal colonization by instilled wild-type S. aureus SA113 that is not antibiotic resistant from indigenous coagulase-negative staphylococci (CoNS), a subtractive technique was used. Supernatant from dissected noses instilled with wild-type SA113 was plated on TSA plus 7.5% NaCl with and without 1 μg/ml lysostaphin. This concentration of lysostaphin inhibited growth of S. aureus but not CoNS from cotton rat noses (45). Nasal colonization was determined as the CFUs on TSA without lysostaphin minus the CFUs on TSA with lysostaphin. All guidelines of both the USDA and the Biosynexus IACUC were followed during the animal studies described in this application.

Preparatory experiments demonstrated that elimination of the natural nasal flora with streptomycin or nafcillin prior to instillation did not considerably affect subsequent nasal colonization by S. aureus. Moreover, S. aureus nasal colonization had no apparent influence on the natural flora.

In the experiment using the complemented ΔtagO mutant, both the mutant and the complemented mutant were instilled in PBS suplemented with antibiotic (2.5 μg/ml erythromycin or 101 μg/ml chloramphenicol respectively) to maintain selective pressure for the plasmid baring the tagO gene.

In order to study the alleviation of nasal colonization by WTA, cotton rat noses were preinstilled with purified WTA five minutes before instillation with bacteria in three different experiments. Comparatively low numbers of bacteria were used to permit an efficient competition by the preinstilled WTA. Either S. aureus SA113 wild-type (3×10⁴ CFU) or the clinical isolate MBT 5040 (5×10⁵ CFU), which was easy to identify on agar plates because of its streptomycin resistance (45), were used in experiments 1 or 2 and 3, respectively. WTA from SA113 wild-type in 10 μl PBS (50 μg in experiment 1 and 3 or 200 μg in experiments 2) were applied. In experiment 1, treatment with 50 μg WTA was repeated on day one and two. Ten, six, and twenty animals were used in experiments one, two, and three, respectively. In each experiment, 50% of the animals were pretreated with WTA in PBS or with PBS alone as a control.

Sensitivity studies with hNP1-3, LL-37, and lactoferrin: In order to prepare bacteria for sensitivity experiments, Mueller Hinton Broth was inoculated with 1/100 volumes of an overnight bacterial culture and shaken vigorously at 37° C. until mid-logarithmic phase was reached. The bacteria were washed thrice with potassium phosphate buffer (10 mM, pH 7.5) containing 0.05% HSA. Bacteria at a concentration of 1×10⁶ CFU/ml were incubated with 100 μg hNP1-3/ml, 10 μl LL-37/ml, or 500 μg lactoferrin/ml in the same buffer after preheating all compounds at 37° C. Samples of 10 μl each were shaken at 37° C. and killing was stopped after various time points by 25-fold dilution in ice-cold potassium phosphate buffer. Viable bacteria were counted 24 h after plating appropriate dilutions on LB agar. Human lactoferrin was purchased from Sigma (Saint Louis, Mo.). hNP1-3 was purified from human granulocytes as described recently (63). LL-37 was synthesized by solid-phase peptide synthesis using the Fmoc/Bu^{t} strategy and a polystyrene resin with a Rink amide resin (2). After deprotection the peptide was purified by reverse-phase preparative HPLC on a Nucleosil C4 column (150×10 mm). The purity of the peptide was checked by analytical reverse-phase HPLC and the preparation was found to be 97% pure. The peptide identity was confirmed by electrospray mass spectrometry and MALDI-MS. Equal numbers of S. aureus Sal 13 wild-type, ΔtagO, and ΔdltA bacteria were incubated with 100 μg/ml of human defensins hNP1-3, x μg/ml cathelicidin LL-37, or 500 μg/ml lactoferrin and viable bacteria were counted after different times of incubation.

Adherence to epithelial cells: An established human alveolar epithelial cell line A549 (45) was cultured in Dulbecco's modified Eagle's medium Nut mix F-12 (DMEM-F12) (Gibco-BRL, Carlsbad, Calif.), supplemented with 10% heat-inactivated fetal bovine serum (Biochrome, Berlin, Germany) and 2 mM glutamine. Primary human bronchial epithelial cells (NHBE) and the required medium components were purchased from Clonetics (Walkersville Md.). They were cultured in bronchial epithelial cell growth medium (BEGM) supplemented with the BulletKit according to the manufacturers instructions and used up to passage number four. Primary human nasal epithelial cells (HNEC) and the required medium components were purchased from Oligene (Berlin, Germany). HNECs were cultured according to the manufacturer's instructions and used up to passage number four. All types of etpithelial cells were seeded to 24-well culture plates at numbers of 5×10⁴/well (A549), 2×10⁴/well (NHBE), or 1×10⁴/well (HNEC) and incubated at 37° C. under 5% CO₂. When confluent, the monolayers were washed three times with RPMI 1640 medium (Sigma) and used for adhesion assays.

In order to prepare bacteria for adherence experiments, Mueller-Hinton broth was inoculated with 0.01 volumes of an overnight bacterial culture and shaken vigorously at 37° C. until the mid-logarithmic phase was reached. The bacterial pellet was washed three times, and resuspended in PBS. Subsequently, 0.1 mg/ml of fluorescein isothiocyanate (FITC) was added and the bacteria were labeled at 37° C. for 1 h. After three washes with PBS, the bacteria were resuspended in RPMI and adjusted to the same cell number using a Neubauer chamber.

Bacterial adhesion assays were carried out using the following standardized protocol: Confluent epithelial cell monolayers grown in 24-well multiwell plates (approximately 6.4-7×10⁴ NHBE cells/well; 7×10⁵ A549 cells/well; 4×10⁴ HNEC cells/well) were washed twice with RPMI and inoculated with FITC-labeled bacteria suspended in RPMI. Dose dependency of bacterial adherence was confirmed by using increasing multiplicities of infection (MOI), ranging from 5 to 100 (data not shown) and MOIs of 50 or 100 were eventually used for A549 and HNECs or NHBE cells, respectively. After incubation for 1 hour at 37° C. under 5% CO₂, the wells were washed three times with RPMI and cells were fixed with 3.5% paraformaldehyde in PBS. No morphological changes in the cells were observed after this procedure in control wells containing RPMI without bacteria. Adherent bacteria/mm² were counted using a Leica DMRIBDE fluorescence microscope with a PL Fluotar L 63×0.7 objective (Leica Microsystems, Wetzlar, Germany). Experiments were run in triplicate and up to 10 random fields were counted in each well. Under these conditions, 10.0 plus or minus 1.0% of the applied S. aureus wild-type cells adhered to HNEC (mean and SD of 5 experiments).

To evaluate the effect of purified WTA on adhesion of S. aureus to epithelial cells, the following modifications were made. Confluently grown A549 or HNEC cells were preincubated with different concentrations of purified WTA (125 μg/ml, 250 μg/ml, or 500 μg/ml) of S. aureus SA113 wild-type or ΔdltA dissolved in RPMI. In the case of the ΔtagO mutant, equal volumes of samples prepared by the same method but lacking WTA were applied to the epithelial cells. After one hour of incubation the cells were washed and inoculated with FITC-labeled bacteria as described above.

IL-8 Induction: IL-8 induction was studied by incubating HNECs with S. aureus strains under conditions described above for adherence studies except that the bacteria were not labeled and were inactivated after one hour by addition of gentamycin (100 μg/ml) followed by incubation for an additional 8 hours. IL-8 was quantified by ELISA (R&D Systems, Minneapolis, Minn.).

Adherence of WTA-coated microspheres: In order to coat amine-modified fluorescent microspheres (FluoSpheres, 1.0 μm diameter, yellow-green fluorescent, Molecular Probes, Eugene, Oreg.) with WTA, the beads were washed with potassium phosphate buffer (PPB) (10 mM, pH 7.5) and were incubated for 30 minutes at room temperature with 200 μl WTA (500 μg/ml) under slow shaking. WTA had been ethanol-precipitated and dissolved in PPB. The WTA-coated beads were washed twice and resuspended in PPB containing 1% BSA (Sigma) to block hydrophobic areas. The amount of adsorbed WTA was determined by measuring the amount of GlcNAc released by boiling 100 μl of WTA-coated beads without BSA at 100° C. for 10 minutes. The WTA-coated beads were diluted in RPMI, adjusted to defined concentration using a Neubauer chamber, and the various samples were tested for equal fluorescence. Samples were used in adhesion assays on confluently grown A549 cells with MOIs of 50, 25 and 12.5 or on HNECs with MOIs of 60, 30, and 15 as described for FITC-labeled bacteria. The relative fluorescence at 505/515 nm per well was quantified with a fluororeader (FL600, Bio-TEK Instruments, Winooski, Vt.). For evaluation of the results the number of beads/mm² epithelial cells was determined in some experiments by counting as described above for FITC labeled bacteria (i.e., counted microscopically).

Adherence to Fibronectin (Fn): Bacterial adherence to solid-phase Fn was studied as described by Wolz et al. (93). Briefly, 96-well microtiter plates (Costar, Acton, Mass.) were coated with 20 μg Fn/well in 50 mM sodium carbonate buffer (pH 9.6) for 15 hours at 4° C. Subsequently, wells were blocked with 3% BSA in TBS (25 mM Tris-HCl, 100 mM NaCl, pH 7,5) for two hours and washed twice with TBS. Bacteria were grown in IMDM to mid-logarithmic phase, washed twice with TBS, and adjusted to 1×10⁹ cells/ml using a Neubauer chamber. 200 μl of bacterial suspensions were added to each well. After 1 h incubation at 37° C. the wells were washed three times with TBS, stained with safranin for 1 min, and A₄₉₂ was determined in a micro plate reader (SpectraMAX 360 pc, Molecular Devices, Sunnyvale, Calif.).

Killing studies with hNP1-3, LL-37, and lactoferrin: In order to prepare bacteria for killing experiments, Mueller Hinton Broth was inoculated with 1/100 volumes of an overnight bacterial culture and shaken vigorously at 37° C. until mid-logarithmic phase was reached. The bacteria were washed thrice with potassium phosphate buffer (10 mM, pH 7.5) containing 0.05% HSA. Bacteria at a concentration of 1×10⁸ CFU/ml were incubated with 100 μg hNP1-3/ml, 500 μg lactoferrin/ml, or 100 μl LL-37/ml in the same buffer after preheating all compounds at 37° C. Samples of 10 μl each were shaken at 37° C. and killing was stopped after various time points by 25-fold dilution in ice-cold potassium phosphate buffer. Viable bacteria were counted 24 h after plating appropriate dilutions on LB agar. Human lactoferrin was purchased from Sigma. hNP1-3 was purified from human granulocytes as described recently (18, 68). LL-37 was synthesized by solid phase peptide synthesis and purified by reverse-phase HPLC. The amino acid sequence of LL-37 was as follows: LLGDFFRKSKEKIGKEFKRIVQRIKDFLRNLVPRTES (SEQ ID NO. 3). The purity and identity of the peptides were confirmed by HPLC and mass spectrometry. Lactoferrin was purchased from Sigma.

Mutant sensitivity to lysostaphin: In order to compare the activity of lysostaphin towards S. aureus wild type and ΔtagO, B-media was inoculated with 1/100 volumes of an overnight culture and shaken at 37° C. until mid-logarithmic phase was reached. The bacteria were washed three times in PBS. All steps were performed at 4° C. Bacteria at an A₆₀₀ of 1 were incubated in PBS at 30° C. for 1 hour with or without 1 μg/ml of lysostaphin (Merck). The decrease of the A₆₀₀ was measured with a micro plate reader (SpectraMAX 360 pc, Molecular Devices, Sunnyvale, Calif.) every 10 minutes.

The invention, having been described above, may be better understood by reference to examples. The following examples are intended for illustration purposes only, and should not be construed as limiting the scope of the invention in any way.

### EXAMPLE 1

### Generation of and Characterization of WTA Deficient S. aureus

Colonization of the anterior nares in 30-40% of the human population is a major risk factor for developing severe Staphylococcus aureus infections. The mechanisms which mediate adherence and survival in the nares, however, remain unknown.

The present invention defines the first mutant deficient in production of wall teichoic acids (WTA), surface-exposed staphylococcal cell wall polymers, and demonstrate that WTA is important for nasal colonization in a cotton rat model. WTA deficiency did not affect susceptibility to defensins and other nasal antimicrobial molecules, but it did abrogate adherence to human airway epithelial cells. These data shed new light on the molecular basis of nasal colonization by S. aureus and provides strategies for preventing and combating S. aureus infections.

Staphylococcus aureus is one of the most virulent human bacterial pathogens in terms of frequency and severity of blood stream infections and sepsis, along with metastatic, often chronic infections and high a mortality rate. The recent increase in the incidence of multiple antibiotic-resistant isolates of S. aureus, including the emergence of vancomycin-resistant strains, has raised the specter of untreatable S. aureus infections and increases the urgency for the development of novel preventive and anti-infective strategies (40). Accordingly, one of the major risk factors for S. aureus infections—nasal carriage—has gained increasing interest (90) but the molecular basis of adherence to and multiplication on nasal epithelia has remained elusive. Several studies have documented that ˜20% of the healthy adult population are persistent S. aureus carriers while another 60% are intermittently colonized in the anterior nares. This carrier status is associated with an increased risk of S. aureus infections in patients undergoing surgery or on dialysis and in those infected with HIV (65) to name a few. Accordingly, great efforts are made to eliminate S. aureus from the noses of patients and health-care workers. The antibiotic mupirocin has been effective for topical eradication of S. aureus and can reduce the risk of S. aureus infections but the increasing prevalence of mupirocin resistance demands new strategies to interfere with nasal colonization by S. aureus (51).

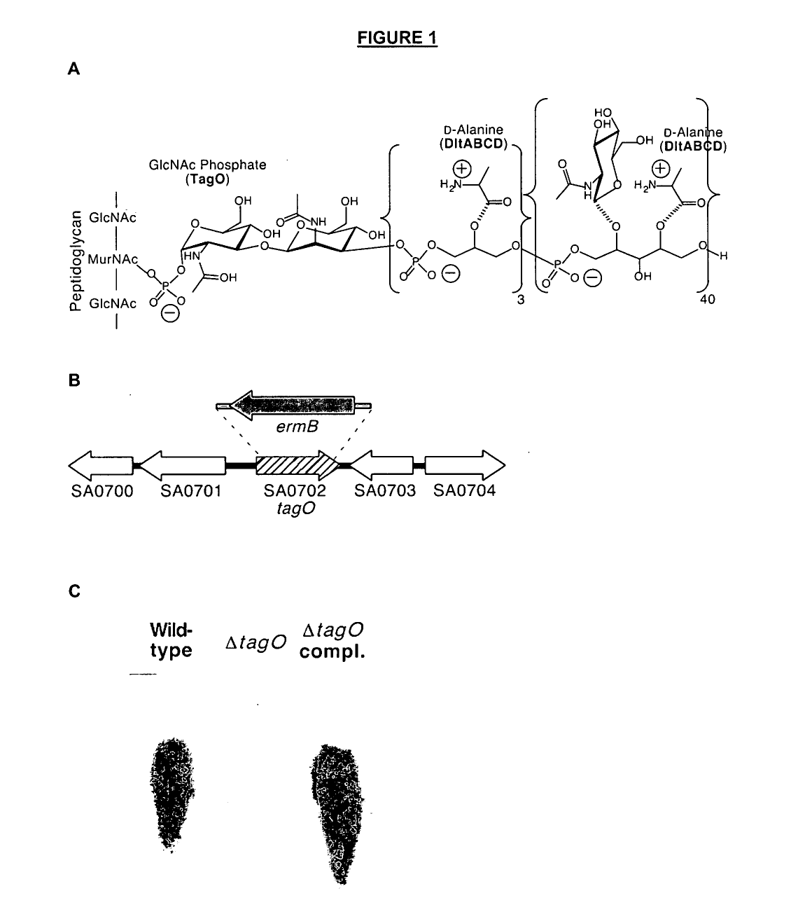

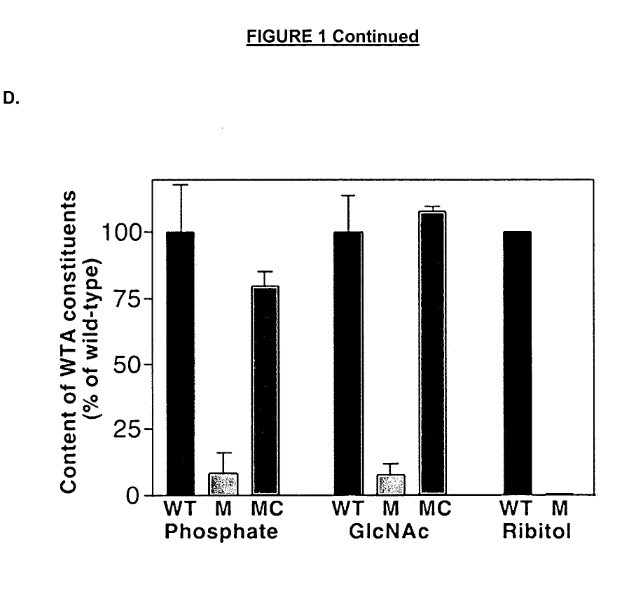

The staphylococcal cell envelope contains in addition to surface proteins, teichoic acids, which are complex surface-exposed polymers, whose role in bacterial pathogenesis and physiology are not yet fully understood and whose biosynthesis has attracted only limited attention (31, 58, 71). Teichoic acids are either covalently linked to the peptidoglycan (wall teichoic acids, WTA) or connected to membrane glycolipids (lipoteichoic acids, LTA) (31). The failure of gene replacement studies in Staphylococcus epidermidis (32) and Bacillus subtilis (53) has led to the assumption that WTA represents an important component of all Gram-positive bacteria; nevertheless, a WTA-deficient S. aureus mutant was described in the late 1960s (14). Due to lack of information about the mutated gene and possible rates of reversion, however, the relevance of this study has been questioned. S. aureus produces a WTA distinct from those of all other a staphylococcal species. This unique structure may be involved in virulence mechanisms of S. aureus. This unique WTA is composed of −40 ribitolphosphate repeating units and modified with N-acetylglucosamine (GlcNAc) and D-alanine (31) (FIG. 1A). A S. aureus mutant lacking the D-alanine modifications in WTA and LTA has been recently described (38). Characterization of this mutant demonstrated key roles of the alanine modifications in the escape from killing by defensins and other cationic host defense factors (68), in biofilm formation (38), and in survival in a mouse sepsis model (18).

Generation of the WTA Deficient Mutant

In order to more thoroughly study the role of teichoic aids in virulence, we identified the WTA-biosynthetic gene tagO, and generated the first defined WTA-deficient mutant. As discussed in the subsequent Examples below, this defined mutant (ΔtagO) demonstrates the role of WTA in nasal colonization by S. aureus in a cotton rat model.

Previous studies in B. subtilis have led to identification of some of the genes for WTA biosynthesis by generating conditional mutants (32, 46, 72, 83, 95). However many genes of the biosynthetic pathway have remained uncharacterized (71). According to biochemical studies (9, 17, 95), the first enzyme should be a membrane protein which transfers GlcNAc from UDP-GlcNAc to the universal bacterial lipid carrier, bactoprenol. A B. subtilis gene with homology to this type of enzyme, tagO, has recently been shown to be important for viability and repression of this gene's transcription has led to reduced incorporation of radioactive WTA precursors into the cell wall (82). We have now identified related genes in the genomes of S. aureus and all Gram-positive bacteria producing WTA (data not shown).

The S. aureus tagO homologue was replaced by an erythromycin resistance cassette (FIG. 1B) in S. aureus Sal 13 to evaluate its possible involvement in WTA biosynthesis. Cell walls from wild-type S. aureus Sal 13 and the ΔtagO mutant were prepared and WTA was released by acidification. WTA was undetectable in the ΔtagO mutant by polyacrylamide gel electrophoresis but WTA reappeared upon complementation with plasmid the pRBtagO bearing the tagO gene (FIG. 1C). Analysis of the phosphate and GlcNAc contents of the ΔtagO mutant revealed only trace amounts of wild-type phosphate (8.25%) and GlcNAc (8.14%) (FIG. 1D). These trace amounts are probably derived from residual nucleic acid and peptidoglycan contamination. Ribitol, the hallmark of WTA, was nearly undetectable in samples from the mutant (168 nmol ribitol in the wild-type versus 0.76 nmol ribitol in the mutant per mg cell wall dry weight) (FIG. 1D). This residual amount of ribitol was close to the detection limit and may represent residual impurities in the gas chromatography capillary from previous runs of other samples. Several S. aureus phages such as 3A52 and φ11 are known to employ WTA as a receptor for infection of bacterial cells (61, 63). The tagO mutant was totally resistant to these phages while the wild-type strain and the complemented mutant were susceptible to phage infection (data not shown). Taken together, these data demonstrate that the tagO mutant is devoid of WTA. The similarity of TagO to UDP-N-acetylglucosamine transferases (82) suggests a role in the first step of WTA synthesis, the transfer of GlcNAc to the bacterial lipid carrier, bactoprenol.

Initial Characterization of the WTA Deficient Mutant

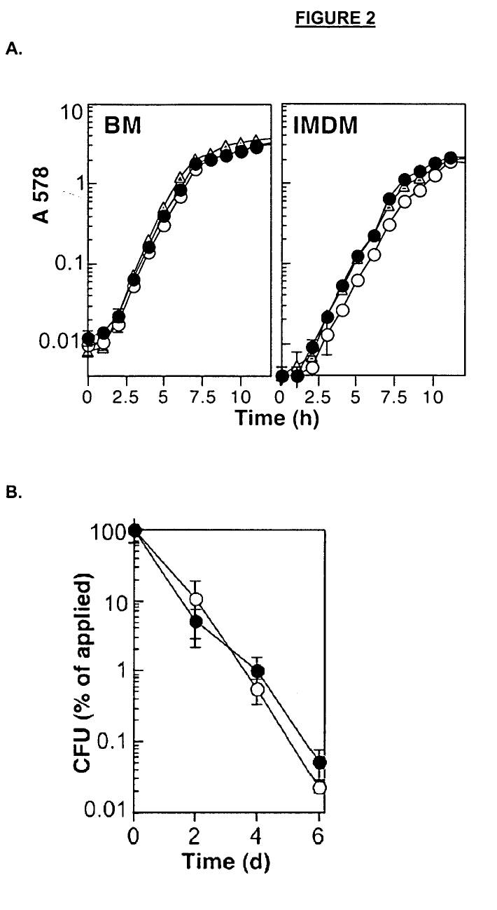

Patterns of cell wall-anchored proteins in wild-type and ΔtagO showed no major differences (data not shown). Growth characteristics of S. aureus Sal 13 wild-type and the ΔtagO mutant were compared in a complex media (Basic Medium, BM) and a minimal media (Iscove's modified Dulbecco's Medium, IMDM). Generation times were very similar for both strains under conditions relevant in the anterior nares (30° C., good aeration) (FIG. 2A), indicating that WTA plays only a minor role in S. aureus cell replication. The ΔtagO mutant had only a slightly increased lag phase and reduced generation-time at 37° C. in rich medium (61±3 min in the mutant vs. 50±4 min in the wild-type; mean±SD). Survival rates of wild-type and ΔtagO in the stationary phase were very similar over a period of at least six days (FIG. 2B).

### EXAMPLE 2

### WTA is Important for Adherence to Epithelial Cells

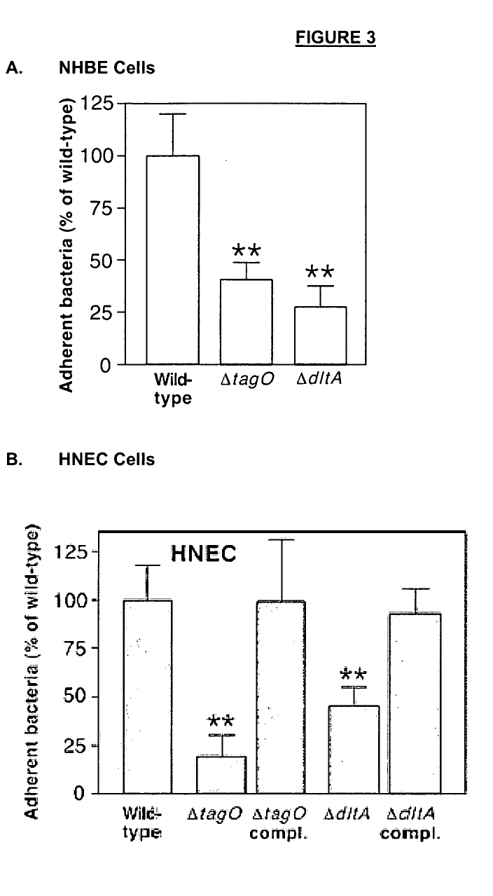

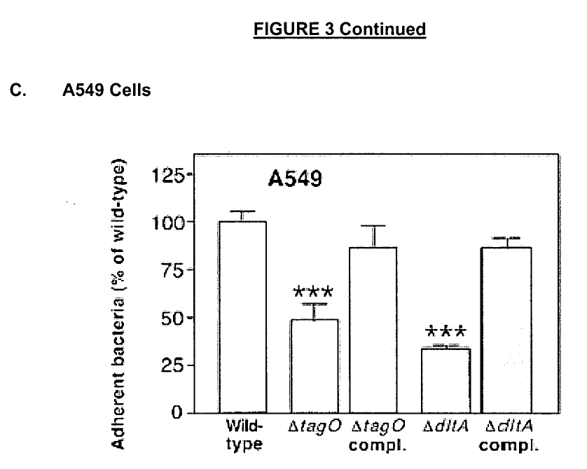

Another mutant of S. aureus SA113, ΔdltA, has been described (68). This mutant lacks the D-alanine modifications in WTA and in the membrane-anchored lipoteichoic acid (FIG. 1A). The ΔtagO and ΔdltA mutants were analyzed for their capacity to adhere to human airway epithelial cells (normal human bronchial epithelial cells, NHBE) (FIG. 3A) and human nasal epithelial cells (HNEC) (FIG. 3B). In HNECs, both mutants adhered considerably less efficiently than the wild-type (ΔtagO, 80% reduced adherence; ΔdltA, 55% reduced adherence; FIG. 3B). Mutant strains complemented with the missing gene show wild-type levels of adhesion. Similar but less pronounced differences were found when adherence to primary human bronchial cells were analyzed (FIG. 3A).

The ΔtagO and ΔdltA mutants were also analyzed for their capacity to adhere to the human airway epithelial cell line A549 (FIG. 3C). Both mutants adhered considerably less efficiently than the wild type strain (ΔtagO, 51% reduced adherence; ΔdltA, 66% reduced adherence; data set #1) while the complemented strains of each mutant exhibited wild-type levels of adhesion.

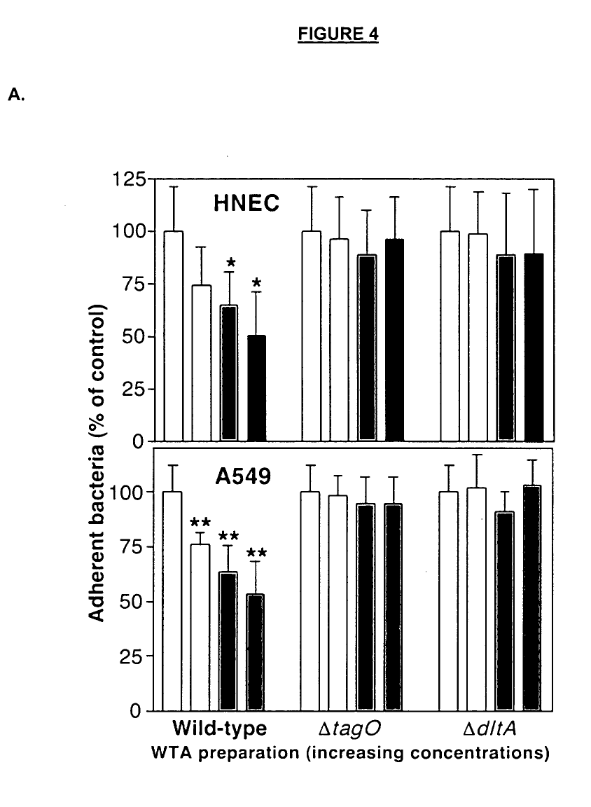

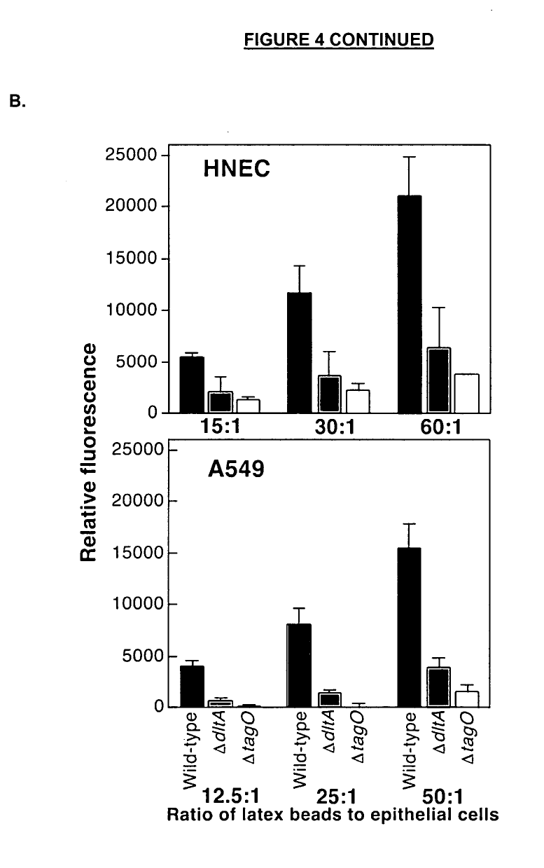

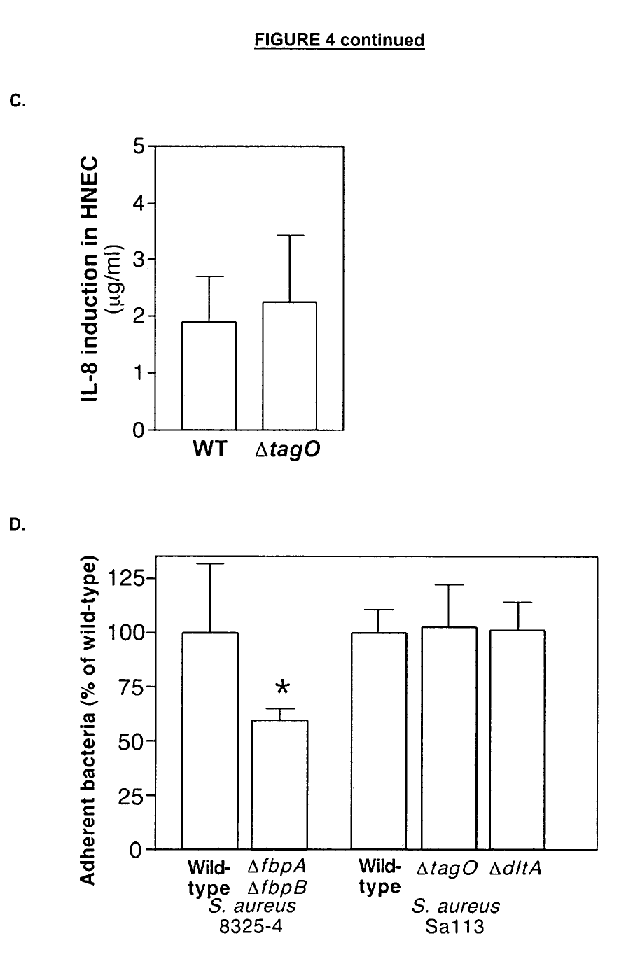

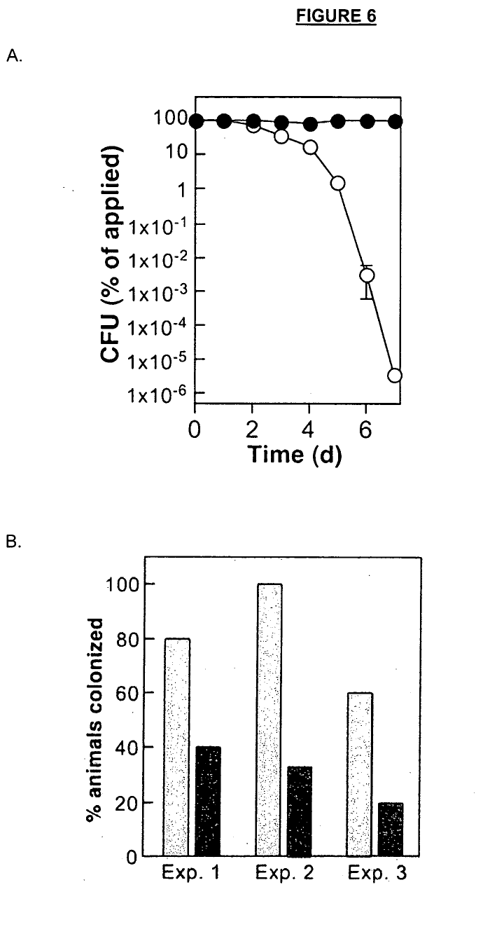

Preincubation of A549 cells with WTA isolated from wild-type S. aureus caused a dose-dependent reduction in numbers of wild type S. aureus bound to the cells (FIG. 4A). When WTA preparations from the dltA mutant or preparations from the tagO mutant that had been prepared by the same method but were lacking WTA were used in the same assay however, no such reduction in binding of wild type S. aureus to the eukaryotic cells was observed. Latex beads coated with wild-type WTA showed a strong, dose-dependent increase in binding to A549 compared to non-coated beads, while coating with samples from ΔtagO or ΔdltA caused no or only very weakly increased binding (FIG. 4B). This suggests that there is a specific interaction between wild-type WTA with epithelial cells, which is further substantiated by a pronounced reduction in the capacity of S. aureus to colonize cotton rat noses upon preinstillation of the noses with WTA (FIG. 6B).

There were no significant differences in the induction of proinflammatory cytokines by S. aureus wild-type, ΔtagO, and ΔdltA in A549 (FIG. 4C) indicating that the proinflammatory capacities of the three strains are similar.

Fibronectin- (Fn-) mediated interactions play a role in S. aureus binding to human cells (24) and S. epidermidis WTA has been shown to enhance adhesion to immobilized Fn (41). However, ΔtagO and ΔdltA did not show any reduction in their in vitro capacity to bind to Fn (FIG. 4D), indicating that interactions other than those involving Fn are responsible for WTA-mediated binding of S. aureus to epithelial cells.

### EXAMPLE 3

### Susceptibility of Mutants to Antimicrobial Peptides and Lysostaphin

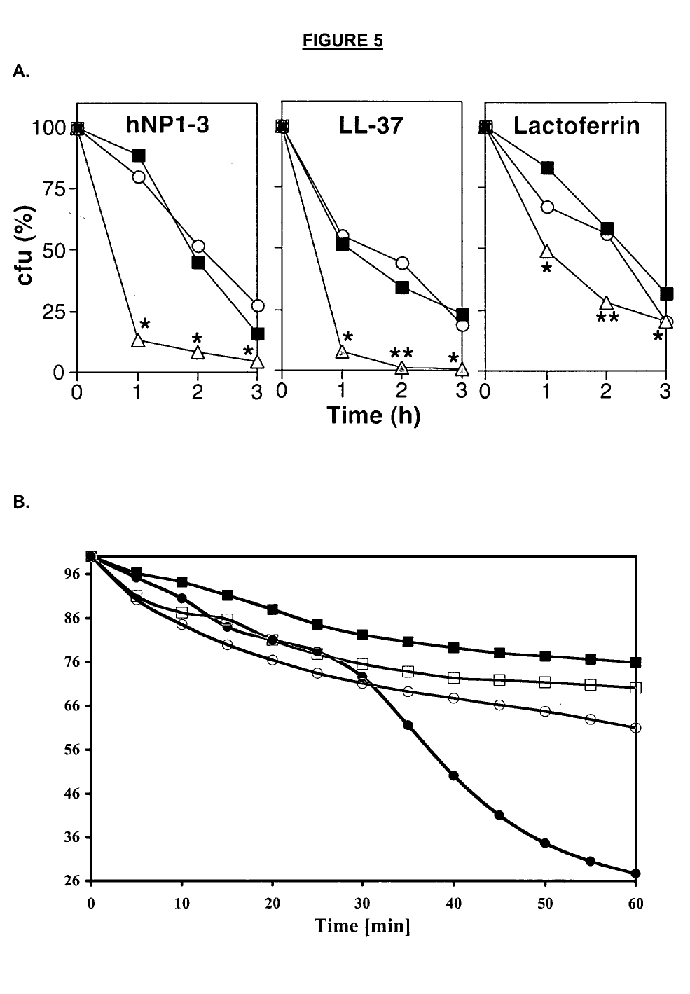

Nasal secretions from humans and rodents contain a number of cationic antimicrobial peptides and proteins (16, 89). Airway epithelia from humans and rodents produce a number of antimicrobial substances including defensins, cathelicidins, lactoferrin, and others. The teichoic acid D-alanine esters play a key role in S. aureus resistance to this class of host defense factors (68, 18). In addition, Fn-mediated interactions with integrins have been shown to play a role in S. aureus binding to epithelial cells (25). As demonstrated in Example 4 below, the capacity of ΔtagO and ΔdltA to colonize cotton rat noses is decreased in comparison to wild type. This may might be due to increased susceptibility of the mutants to these innate host defense factors. Accordingly, susceptibilities of S. aureus wild-type, ΔdltA, and ΔtagO to human defensin hNP1-3, cathelicidin LL-37, and lactoferrin were compared (FIG. 5A). LL-37 was the most potent compound while 10- or 50-fold higher amounts were necessary to achieve similar antibacterial activities with hNP1-3 and lactoferrin, respectively. The Ad/LA mutant was considerably more susceptible to all three substances. In contrast, the lack of WTA in ΔtagO had no influence on the activity of the three peptides as compared to the wild-type strain (FIG. 5A). This result indicates that an increased susceptibility of S. aureus ΔtagO to nasal patient defenses is probably not responsible for the inability of this mutant to colonize cotton rat noses.

Susceptibilities of S. aureus wild-type (solid symbols) and ΔtagO (open symbols) to lysostaphin were also compared (FIG. 5B). Bacterial suspension with an A₆₀₀ of 1 were incubated at 30° C. for 1 hour with (circles) or without (squares) lysostaphin at a concentration of 1 μg/ml. The values in FIG. 5B are given as percentages of the initial A₆₀₀. The WTA deficient mutant (ΔtagO) had increased resistance to lysis with lysostaphin as compared to wild type. Indeed, the addition of lysostaphin to the ΔtagO bacterial culture made little difference in the amount of bacterial remaining after a 1 hour incubation.

### EXAMPLE 4

### Role of WTA in Nasal Colonization

Indirect data from the 1980s have implicated teichoic acids in binding to human airway epithelial cells (2) suggesting a possible role of WTA in nasal colonization. The recently developed cotton rat (Sigmadon hispidis) model of S. aureus nasal colonization permits consistent and persistent high-level nasal colonization (45). The cotton rat model reflects well the situation in human S. aureus carriers since the cotton rat nares have similar histological properties to humans with squamous, cuboid to columnar, columnar, and pseudostratified columnar epithelial areas (73)3 Cotton rats have been shown to be susceptible to many bacterial and viral respiratory human pathogens and to follow disease courses similar to those observed in humans (60).

Six week-old female cotton rats were intranasally instilled with equal numbers of either S. aureus SA113 wild type or a defined SA113 mutant as shown in Table 1. After seven days, nasal colonization was enumerated.

TABLE 1

| | Number of rats | | |
|---|---|---|---|
| | colonized/number | | Median |
| Strain | tested^{a} | Mean CFUs^{b} | CFUs^{b} |
| Wild Type | 15/15 (3) | 6011 | 6207 |
| ΔtagO | 0/15 (3)^{c} | 0 | 0 |
| ΔtagO | 5/5 (1)^{c} | 173 | 146 |
| complemented | | | |
| ΔdltA | 4/10 (2)^{c} | 30 | 33 |

^{a}Numbers in parenthesis reflect the number of experiments.
^{b}Colony Forming Units (CFUs) recovered per colonized nare.
^{c}In one experiment, bacteria were instilled in PBS containing antibiotics as described in Methods.

While the noses of all animals instilled (15 out of 15) with wild-type bacteria were colonized with an average of 6011 CFUs/nose, no S. aureus bacteria were detectable in the noses of ΔtagO-instilled cotton rats.

In a follow-up experiment, nasal colonization by ΔtagO complemented with plasmid pRBtagO was examined. Five of five animals were colonized with the complemented mutant seven days after instillation (Table 1), albeit at a lower level than with wild-type bacteria. This lower level of colonization can be explained by the lack of antibiotic selective pressure in the nose, resulting in loss of the complementing plasmid. This explanation is supported by the finding that pRBtagO in vitro (FIG. 6A). It should be noted that all bacteria recovered from animals instilled with the complemented mutant had retained the plasmid, indicating that the presence of WTA is a prerequisite for continued nasal colonization.

Most of the ΔdltA-instilled animals were also devoid of S. aureus, only 4 of 10 cotton rats were nasally colonized and these four animals had a low average of 33 CFUs/nose. These data indicate that there is an important role for intact teichoic acids for nasal colonization by S. aureus.

In another experiment, the time course of nasal colonization by S. aureus wild-type and ΔtagO was compared. One and two days after bacterial instillation, nasal colonization in ΔtagO-instilled animals was 90.7±1.4% and 98.3±0.3% lower, respectively (mean and SD of at least ten animals infected with wild-type or ΔtagO), as compared to wild-type-instilled animals, which indicates that WTA deficiency resulted in a rapid elimination of the bacteria from cotton rat noses.

The effect of precoating the nares of cotton rats with WTA before exposure to S. aureus was determined. There was a pronounced reduction in the capacity of S. aureus to colonize cotton rat noses upon preinstillation of the noses with WTA (FIG. 6B; compare PBS only (light gray bars) to WTA treated (dark gray bars)). Thus, treating cotton rat nares with a WTA preparation before introduction of S. aureus into the nares alleviated the staphylococcal infection.

### EXAMPLE 5

### Antibodies that Specifically Bind to WTA

Antibodies that specifically bind WTA are generated by methods well known to the skilled artisan. See U.S. Pat. No. 6,610,293, which is incorporated herein by reference. For example, polyclonal antibodies that specifically recognize WTA are generated by inoculating mice subcutaneously with a WTA preparation. The WTA preparation is comprised of WTA or WTA fragments and complete Freund's adjuvant. A range of antigen amounts are administered, for example, 10 μg, 50 μg, or 100 μg. This initial inoculation is followed by one or more boosting inoculations at intervals of approximately 1 to 2 months. The boosting antigen preparation comprises WTA or WTA fragments in incomplete Freund's adjuvant. Different combinations of antigen amounts and boosting schedules are carried out to determine the dosage and boosting schedule that will produce the optimal immune response to WTA. Polyclonal antibodies are prepared by harvesting the blood of the vaccinated mice and preparing blood serum by centrifuging the blood sample to separate serum from cellular components. Polyclonal antibodies may also be produced in rabbits by similar methods.

Monoclonal antibodies are prepared by removing the spleens of the vaccinated mice and producing hybridoma cell lines from the harvested splenocytes. For example, hybridomas are prepared by general methods (6, 79). Generally, a total of 2.135×10⁸ spleenocytes from a vaccinated mouse are mixed with 2.35×10⁷ SP2/0 mouse myeloma cells (ATCC Catalog number CRL1581) and pelleted by centrifugation (400×g, 10 minutes at room temperature) and washed in serum free medium. The supernatant is removed to near-dryness and fusion of the cell mixture is accomplished in a sterile 50 ml centrifuge conical by the addition of 1 ml of polyethylene glycol (PEG; mw 1400; Boehringer Mannheim) over a period of 60-90 seconds. The PEG is diluted by slow addition of serum-free medium in successive volumes of 1, 2, 4, 8, 16 and 19 mis. The hybridoma cell suspension is gently resuspended into the medium and the cells are pelleted by centrifugation (500×g, 10 minutes at room temperature). The supernatant is removed and the cells are resuspended in medium RPMI 1640, supplemented with 10% heat-inactivated fetal bovine serum, 0.05 mM hypoxanthine and 16 uM thymidine (HT medium). One hundred μl of the hybridoma cells are planted into 760 wells of 96-well tissue culture plates. Eight wells (column 1 of plate A) receive approximately 2.5×10⁴ SP2/0 cells in 100 μl. The SP2/0 cells serve as a control for killing by the selection medium that is added 24 hours later.

Twenty four hours after preparation of the hybridomas, 100 μl of RPMI 1640, supplemented with 10% heat-inactivated fetal bovine serums, 0.1 mM hypoxanthine, 0.8 uM aminopterin and 32 uM thymidine (HAT medium) is added to each well. Ninety six hours after the preparation of the hybridomas, the control SP2/0 cells are checked for cell death, indicating that the HAT selection medium had successfully killed the unfused SP2/0 cells.

Eleven days after the preparation of the hybridomas, supernatants from all wells are tested by ELISA for the presence of antibodies reactive with a WTA preparation. Based on the results of this preliminary assay, cells from 20 wells are transferred to a 24-well culture dish for further growth of positive clones and isolation of MAbs.

Chimeric antibodies may be generated from hybridoma cells by isolating total RNA from these cells, preparing cDNA, and then cloning out the antibody light chain variable region and the antibody heavy chain variable through the use of PCR. See U.S. Pat. No. 6,610,293. Generally, the first strand cDNA synthesis products are purified using a Centricon-30 concentrator device (Amicon). Of the 40 μl of cDNA recovered, 5 μl is used as template DNA for PCR. Typical PCR amplification reactions (100 μl) contain template DNA, 50 pmoles of the appropriate primers, 2.5 units of ExTaq polymerase (PanVera), 1×ExTaq reaction buffer, 200 μM dNTP, 1 mM MgCl₂. The template is denatured by an initial incubation at 96° C. for 5 min. The products are amplified by 15 thermal cycles of 55° C. for 30 sec., 70° C. for 30 sec, then 96° C. for 1 min. followed by 25 step cycles of 70° C. for 1 min., then 96° C. for 1 min. The resulting PCR products are then cloned into a carrier plasmid to facilitate sequencing of the amplified PCR products.

The heavy and light chain variable regions are then subcloned into a mammalian expression plasmid vector for production of recombinant chimeric antibody molecules. The resulting vector expresses both antibody chains with CMV promoter driven transcription. Neomycin resistance serves as a dominant selectable marker for transfection of mammalian cells. For additional detail, see U.S. Pat. No. 6,610,293. Accordingly, humanized antibodies may be produced by techniques well known to those of ordinary skill in the art (8, 39). Human antibodies that bind WTA may be produced by immunizing an animal that produces human antibodies, such as a transgenic mouse that expresses human antibody genes.

The opsonophagocytic bactericidal activity of a preparation of polyclonal antibodies or monoclonal antibodies may be tested by a variety of assays known to the skilled artisan. See U.S. Pat. No. 6,610,293. For example, a neutrophil mediated bactericidal assay may be used. Generally, neutrophils are isolated from adult venous blood by dextran sedimentation and ficoll-hypaque density centrifugation. Washed neutrophils are added to round-bottomed wells of microtiter plates (approximately 10⁶ cells per well) with approximately 3×10⁴ mid-log phase bacteria (i.e., S. aureus). Newborn lamb serum (10 μls), screened to assure absence of antibody to S. epidermidis, is used as a source of active complement.

Forty microliters of immunoglobulin (or serum) are added at various dilutions and the plates are incubated at 37° C. with constant, vigorous shaking. Samples of 10 μls are taken from each well at zero time and after 2 hours of incubation. Each is diluted, vigorously vortexed to disperse the bacteria, and cultured on blood agar plates overnight at 37° C. to quantitate the number of viable bacteria. Opsonophagocytic activity is presented as percent reduction in numbers of bacterial colonies observed compared to control samples.

The ability to systemically alleviate staphylococcal infection may be evaluated in rats. Generally, two day old Wistar rats are injected with −10⁶ S. aureus (type 5, ATCC 12605) subcutaneously just cephalad to the tail. Approximately 30 minutes before and 24 and 48 hours after infection, approximately 320 μg of an anti-WTA MAb is given IP. Control animals are given an equal volume of saline or a control MAb not directed against staphylococci. All animals are observed daily for five days to determine survival.

The ability of an antibody preparation to alleviate staphylococcal colonization in the nares by preinstillation of the MAb may be measured using a technique similar to that described for determining WTA effectiveness. Generally, cotton rat noses are preinstilled with saline or saline containing anti-WTA MAb (2-3 mg purified IgG/mouse dose of 1-3×10⁸ bacteria) five minutes before instillation with bacteria in three different experiments. Comparatively low numbers of bacteria are used to permit an efficient competition by the preinstilled MAbs. Either S. aureus SA113 wild-type (3×10⁴ CFU) or the clinical isolate MBT 5040 (5×10⁵ CFU), which is easy to identify on agar plates because of its streptomycin resistance (45) are used. In each experiment, 50% of the animals are pretreated with WTA in PBS or with PBS alone as a control.

In addition, the effectiveness of a MAb to alleviate an established staphylococcal nasal infection may also be measured. Generally, mice are instilled with 6×10⁷ S. aureus. One and three days following instillation of bacteria, saline or anti-WTA MAb in saline is instilled in the nares of the colonized mice. On day five, mice are sacrificed, the noses prepared as described above, and plated to detect the presence of S. aureus.

### Conclusions

The results of experiments performed demonstrate a significant role of WTA and the d-alanine ester of WTA and/or LTA in nasal colonization. To our knowledge, WTA is the first factor identified as important for nasal colonization. While it may be possible to attribute the lack of nasal colonization by the ΔdltA mutant to this mutant's increased susceptibility cationic antimicrobial components, the ΔtagO mutant was no more susceptible to the antimicrobial components found on nasal epithelia than the S. aureus wild-type. This observation indicates that reasons other than reduced resistance are responsible for the failure of the wall teichoic acid mutants to colonize the cotton rat nares.

Reduced adherence of the ΔtagO mutant to primary epithelial cells and to an epithelial cell line, the WTA-mediated adherence of latex beads, and the dose-dependent reduction of adherence to epithelial cells when preincubated with wild-type WTA but not with mutated WTA indicates that WTA mediates the interaction of S. aureus with airway epithelial cells. These observations also point toward a specific interaction of WTA polymers with patient factors. It remains to be determined whether S. aureus WTA interacts directly with complementary patient cell receptors or whether other molecules are involved in this interaction. Which ever the case, fibronectin and integrins appear not to be directly involved in WTA-mediated attachment of S. aureus to the nasal epithelium. This is in contrast to earlier studies (3) which suggest that teichoic acid is the S. aureus receptor for fibronectin. It has also been suggested (7) that there are two kinds of receptors for S. aureus on nasal cells, one of which is unaffected by teichoic acid. This study argues against this hypothesis or at least suggests that teichoic acid is required for both interactions. Certain mammalian scavenger receptors have been reported to interact with S. aureus cells and purified lipoteichoic acids and some of them have been identified on epithelial cells (66). Whether they are capable of binding WTA and whether these or other receptors are involved in S. aureus binding to nasal epithelia remains to be determined.

The D-alanine esters of teichoic acid appear to play an major but non-important role in nasal colonization. Since both ΔtagO and ΔdltA adhered less efficiently to epithelial cells as compared to wild-type bacteria, the two mutations may interfere with colonization in similar ways. Since ΔdltA is considerably more susceptible to various nasal antimicrobial substances, however, increased inactivation of this strain by nasal patient defenses may contribute to its reduced capacity to colonize cotton rat nares.

Further evidence for a direct role of teichoic acids in adherence to epithelia comes from Listeria monocytogenes whose teichoic acid d-alanine esters have been implicated in binding to epithelial cells (1). Pneumococci, which produce unique choline-containing WTA and LTA bind to endothelial cells by attachment of the choline residues to the PAF receptor (21). Several mammalian lectin-like receptors have been reported to interact with S. aureus cells and purified teichoic acids or similar polymers (62). It is tempting to speculate that receptors of this type mediate S. aureus attachment to nasal cells.

The above experiments shed new light on the molecular interactions between S. aureus and patient cells and on the previously elusive role of WTA in the staphylococcal cell envelope. The various staphylococcal species are very diverse in their WTA structure (26) and these differences may play a role in the tropism of a given species for a certain host organism and for certain areas of skin or mucous membranes. In fact, polyribitolphosphate WTA is unique to S. aureus and is not found in any other staphylococcal species (26). Irrespective of the polymer composition, the linkage unit between cell wall and WTA is conserved and homologues of tagO are found in all WTA-producing Gram-positive bacteria including, listeria, enterococci, streptococci, bacilli, and clostridia. S. aureus may be unique in the dispensability of WTA since S. epidermidis and B. subtilis appear to require its presence for viability (32, 82). Thus, tagO represents an interesting target for new antimicrobial substances that may or may not be bactericidal but may impede their capacity to colonize. Moreover, S. aureus WTA may be considered as a new target for active or passive vaccination.

Having now fully described the invention, it will be appreciated by those skilled in the art that the invention can be performed within a range of equivalents and conditions without departing from the spirit and scope of the invention and without undue experimentation. In addition, while the invention has been described in light of certain embodiments and examples, the inventors believe that it is capable of further modifications. This application is intended to cover any variations, uses, or adaptations of the invention which follow the general principles set forth above.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The following literature references are herein specifically incorporated by reference:

1. Abachin E., C. Poyart, E. Pellegrini et al. 2002. Formation of D-alanyl-lipoteichoic acid is required for adhesion and virulence of Listeria monocytogenes Mol. Microbiol. 43:1-14.

2. Aly, R., H. R. Shinefield, C. Litz, H. I. Maibach. 1980. Role of teichoic acid in the binding of Staphylococcus aureus to nasal epithelial cells. J. Infect. Dis. 141:463-465.

3. Aly, R. and Levitt, S. 1987. Adherence of S. aureus to Squamous Epithelium: Role of Fibronectin and Teichoic Acid. Rev. of Infect. Dis. 9:S341-S350.

4. Augustin, J. & F. Götz. 1990. Transformation of Staphylococcus epidermidis and other staphylococcal species with plasmid DNA by electroporation. FEMS Microbiol. Lett. 66:203-208.

5. Ausubel, F. M. et al. Current Protocols in Molecular Biology (John Wiley and Sons, New York, N.Y. 1990).

6. Bartal and Hirshaut “Current Methods in Hybridoma Formation” in Methods of Hybridoma Formation, Bartal and Heishaut, eds., Humana Press, Clifton, N.J. (1987).

7. Bibel, D. J., R. Aly, H. R. Shinefield, H. I. Maibach, et al. 1982. Importance of the keratinized epithelial cell in bacterial adherence. J. Invest. Derm. 79:250-253.

8. Borrebaeck, Carl A. K. 1995. Antibody Engineering 2^{nd} Ed., Oxford University Press, NY.

9. Bracha, R., Davidson, R., and Mirelman, D. 1978. Defect in biosynthesis of the linkage unit between peptidoglycan and teichoic acid in a bacteriophage-resistant mutant of Staphylococcus aureus. J Bacteriol 134:412-417.

10. Bruckner, R. 1997. Gene replacement in Staphylococcus camosus and Staphylococcus xylosus. FEMS Mircrobiol. Lett. 151:1-8.

11. Chang, F. Y., N. Singh, T. Gayowski, S. D. Drenning, M. M. Wagener and I. R. Marino. 1998. Staphylococcus aureus nasal colonization and association with infections in liver transplant recipients. Transplantation 65:1169-1172.

12. Chapoutot, C., G.-P. Pageaux, P. F. Perrigault, Z. Joomaye, P. Perney, H. Jean-Pierre, O. Jouquet, P. Blanc and D. Larrey. 1999. Staphylococcus aureus nasal carriage in 104 cirrhotic and control patients A prospective study. J. Hepatol. 30:249-253.

13. Chatfield, C., W. O'Neill, R. Cooke, K. McGhee, M. Issack, M. Rahman and W. Noble. 1994. Mupirocin-resistant Staphylococcus aureus in a specialist school population. J. Hosp. Infect. 26:273-278.

14. Chatterjee, A. N. 1969. Use of bacteriophage-resistant mutants to study the nature of the bacteriophage receptor site of Staphylococcus aureus. J. Bacteriol. 98:519-527.

15. Chatterjee, A. N., Mirelman, D., Singer, H. J., and Park, J. T. 1969. Properties of a novel pleiotropic bacteriophage-resistant mutant of Staphylococcus aureus H. J. Bacteriol. 100:846-853.

16. Cole, A. M. et al. 2002. Cationic polypeptides are required for antibacterial activity of human airway fluid. J. Immunol. 169:6985-6991.

17. Coley, J., Archibald, R., and Baddiley, J. 1977. The presence of N-acetylglucosamine 1-phosphate in the linkage unit that connects teichoic acid to peptidoglycan in Staphylococcus aureus. FEBS Lett 80:405-407.

18. Collins, L. V. et al. 2002. Staphylococcus aureus strains lacking D-alanine modifications of teichoic acids are highly susceptible to human neutrophil killing and are virulence attenuated in mice. J. Infect. Dis. 186:214-219.

19. Cookson, B. 1990. Mupirocin resistance in staphylococci. J. Antimicrob. Chemother. 25:497-503.

20. Corbella, X., M. Dominguez, M. Pujol, J. Aytas, M. Sendra, R. Pallares, J. Ariza and F. Gudiol. 1997. Staphylococcus aureus nasal carriage as a marker for subsequent staphylococcal infections in intensive care units patients. Eur. J. Clin. Microbial. Infect. Dis. 16:351-357.

21. Cundell, D. R., N. P. Gerard, C. Gerard, I. Idanpaan-Heikkila, E. I. Tuomanen. 1995. Streptococcus pneumoniae anchor to activated human cells by the receptor for platelet-activating factor. Nature 377:435-438.

22. Dawson, S., L. Finn, J. McCulloch, S. Kilvington and D. Lewis. 1994. Mupirocin-resistant MRSA. J. Hosp. Infect. 28:75-78.

23. Doebbeling, B. N., D. Breneman, H. Neu, R. Aly, B. Yangco, H. Holley, R. Marsh, M. Pfaller, J. McGowan, B. Scully, D. Reagan and R. Wenzel. 1993. Elimination of Staphylococcus aureus nasal carriage in health care workers: analysis of six clinical trials with calcium mupirocin ointment. Clin. Infect. Dis. 17:466-474.

24. Dziewanowska, K. et al. 1999. Fibronectin binding protein and host cell tyrosine kinase are required for internalization of Staphylococcus aureus by epithelial cells. Infect. Immun. 67:4673-4678.

25. Dziewanowska, K. et al., 2000. Staphylococcal fibronectin binding protein interacts with heat shock protein 60 and integrins: role in internalization by epithelial cells. Infect. Immun. 68:6321-6328.

26. Endl, J., Seidl, H. P., Fiedler, F. & Schleifer, K. H. 1983. Chemical composition and structure of the cell wall teichoic acids of staphylococci. Arch. Microbiol. 135:215-223.

27. Entrez Pub Med database for complete S. aureus genome sequences available at

http://www.ncbi.nlm.nih.gov/entrez/query.fcgi?cmd=Retrieve&db=nucleotide&list_uid s=29165615&dopt=GenBank&term=SA0702&qty=1.

28. Exley A. R.; Cohen J.; Buurman W.; Owen R.; Hanson G.; Lumley J.; Aulakh J. M.; Bodmer M.; Riddell A.; Stephens S.; et al. 1990. Monoclonal antibody to TNF in severe septic shock, Lancet 335: 1275-1277.

29. Fattom A.; Shepherd S.; Karakawa W. 1992. Capsular polysaccharide serotyping scheme for Staphylococcus epidermidis, J. Clin. Micro. 30: 3270-3273.

30. Fierobe, L., D. Decre, C. Muller, J.-C. Lucet, J.-P. Marmuse, J. Mantz and J.-M. Demonts. 1999. Methicillin-resistant Staphylococcus aureus as a causative agent of postoperative intra-abdominal infection: relation to nasal colonization. Clin. Infect. Dis. 29:1231-1238.

31. Fischer, W. 1997. Lipoteichoic Acid and Teichoic Acid Biosynthesis: Targets of New Antibiotics? in New targets for new antimicrobial agents R. Hakenbeck, Ed. (Spektrum Akademischer Verlag, Heidelberg, Germany) pp. 47-50.

32. Fitzgerald, S. N. & Foster, T. J. 2000. Molecular analysis of the tagF gene, encoding CDP-Glycerol:Poly(glycerophosphate) glycerophosphotransferase of Staphylococcus epidermidis ATCC 14990. J. Bacteriol. 182:1046-1052.

33. Fleer, A.; Senders R. C.; Visser M. R.; Bijimer R. P.; Gerards L. J.; Kraaijeveld C. A.; Verhoef J. 1983. Septicemia due to coagulase-negative staphylococci in a neonatal intensive care unit: clinical and bacteriological features and contaminated parenteral fluids as a source of sepsis, Pediatr. Infect. Dis. 2: 426-431.

34. Foster, T. J. 1998. Methods Microbiol. 27:433-454.

35. Frebourg, N., B. Cauliez and J.-F. Lemeland. 1999. Evidence for nasal carriage of methicillin-resistant staphylococci colonizing intravascular devices. J. Clin. Micro. 37:1182-1185.

36. Genarro, A. (ed.) 1990. Remington's Pharmaceutical Sciences, 18^{th} Edition, Mack Publishing, Easton, Pa.

37. Greene, C., D. McDevitt et al. 1995. Adhesion properties of mutants of Staphylococcus aureus defective in fibronectin-binding proteins and studies on the expression of fnb genes. Mol. Microbiol. 17:1143-1152.

38. Gross, M. et al. 2001. Key Roll of Teichoic Acid Net Charge in Staphylococcus Aureus Colonization of Artificial Surfaces. Infection Immunology 69:3423-3426.

39. Harlow, Ed; Lane, David. 1988. Antibodies: A Laboratory Manual, Cold Spring Harbor Press, Cold Spring Harbor, N.Y.

40. Hiramatsu, K. 2001. Vancomycin-resistant Staphylococcus aureus: a new model of antibiotic resistance. Lancet Infect. Dis. 1:147-155.

41. Hussain, M., Heilmann, C., Peters, G. & Herrmann, M. 2001. Teichoic acid enhances adhesion of Staphylococcus epidermidis to immobilized fibronectin. Microb. Pathog. 31:261-270.

42. Kluytmans, J., A. van Belkum and H. Verbrugh. 1997. Nasal carriage of Staphylococcus aureus: epidemiology, underlying mechanisms, and associated risks. Clin. Micro. Rev. 10:505-520.

43. Kluytmans, J. A., J. W. Mouton, M. VandenBergh, M.-J. Manders, A. Maat, J. Wagenvoort, M. Michel and H. Verbrugh. 1996. Reduction of surgical-site infections in cardiothoracic surgery by elimination of nasal carriage of Staphylococcus aureus. Infect. Control.Hosp. Epidem. 17:780-785.

44. Kojima Y.; Tojo M.; Goldmann D. A.; Tosteson T. D.; Pier G. B. 1990. Antibody to the capsular polysaccharide/adhesin protects rabbits against catheter-related bacteremia due to coagulase-negative staphylococci, J. Infect. Dis. 162: 435-441.

45. Kokai-Kun, J. F., S. M. Walsh, T. Chanturiya, J. J. Mond. 2003. Lysostaphin cream eradicates Staphylococcus aureus nasal colonization in a cotton rat model. Antimicrob. Agents. Chemother. 47:1589-1597.

46. Lazarevic, V., and Karamata, D. 1995. The tagGH operon of Bacillus subtilis 168 encodes a two-component ABC transporter involved in the metabolism of two wall teichoic acids. Mol Microbiol 16:345-355.

47. Lee, J. C. 1996. The prospects for developing a vaccine against Staphylococcus aureus, Trends in Micro. 4:162-66.

48. Lee, Y.-L., T. Cesario, A. Pax, C. Tran, A. Ghouri and L. Thrupp. 1999. Nasal colonization by Staphylococcus aureus in active, independent community seniors. Age Ageing. 28:229-232.

49. LoBuglio A. F.; Wheeler R. H.; Trang J.; Haynes A.; Rogers K.; Harvey E. B.; Sun L.; Ghrayeb J.; Khazaeli M. B. 1989. Mouse/human chimeric monoclonal antibody in man: kinetics and immune response, P.N.A.S. 86: 4220-4224.

50. Majcherczyk, P. A., Rubli, E., Heumann, D., Glauser, M. P., and Moreillon, P. 2003. Teichoic acids are not required for Streptococcus pneumoniae and Staphylococcus aureus cell walls to trigger the release of tumor necrosis factor by peripheral blood monocytes. Infect. Immun. 71:3707-3713.

51. Marples, R. R., D. C. E. Speller and B. D. Cookson. 1995. Prevalence of mupirocin resistance in Staphylococcus aureus. J. Hosp. Infect. 29:153-155.

52. Martin, J., F. Perdreau-Remington, M. Kartalija, O. Pasi, M. Webb, J. Gerberding, H. Chambers, M. Tauber and B. Lee. 1999. A randomized clinical trial of mupirocin in the eradication of Staphylococcus aureus nasal carriage in human immunodeficiency virus disease. J. Infect. Dis. 180:896-899.

53. Mauel, C., M. Young, P. Margot, D. Karamata. 1989. The essential nature of teichoic acids in Bacillus subtilis as revealed by insertional mutagenesis. Mol. Gen. Genet. 215:388-394.

54. Mattsson, E., Verhage, L., Rollof, J., Fleer, A., Verhoef, J., and van Dijk, H. 1993. Peptidoglycan and teichoic acid from Staphylococcus epidermidis stimulate human monocytes to release tumor necrosis factor a, interleukin-1§, and interleukin-6. FEMS Immunol. Med. Microbiol. 7:281-288.

55. Merkus, F. W., J. C. Verhoef, N. G. Schipper, and E. Marttin. 1999. Cyclodextrins in nasal drug delivery. Advan. Drug Deliv. Rev. 36:41-57.

56. Mest, D. R., D. H. Wong, K. J. Shimoda, M. E. Mulligan and S. E. Wilson. 1994. Nasal colonization with methicillin-resistant Staphylococcus aureus on admission to surgical intensive care unit increases the risk of infection. Anesth. Analg. 78:644-650.

57. Natsume, H., S. Iwata, K. Ohtak, M. Miyamoto, M. Yamaguchi, K. Hosoya, and D. Kobayashi. 1999. Screening of cationic compounds as an absorption enhancer for nasal drug delivery. Int. J. Pharma. 185:1-12.

58. Neuhaus, F. C., Heaton, M. P., Debabov, D. V., and Zhang, Q. 1996. The dlt operon in the biosynthesis of D-alanyl-lipoteichoic acid in Lactobacillus casei. Microb. Drug Resist. 2:77-84.

59. Nguyen, M. H., C. Kauffman, R. Goodman, C. Squier, R. Arbeit, N. Singh, M. Wagener and V. Yu. 1999. Nasal carriage of and infection with Staphylococcus aureus in HIV-infected patients. Ann. Int. Med. 130:221-225.

60. Niewiesk, S. & G. Prince. 2002. Diversifying animal models: the use of hispid cotton rats (Sigmodon hispidus) in infectious diseases. Lab. Anim. 36:357-372.

61. Novick, R. P. 1991. Genetic systems in staphylococci. Methods Enzymol. 204:587-636.

62. Palaniyar, N., J. Nadesalingam, K. B. Reid. 2002. Pulmonary innate immune proteins and receptors that interact with gram-positive bacterial ligands. Immunobiology 205:575-594.

63. Park, J. T., D. R. Shaw, A. N. Chatterjee, D. Mirelman, T. Wu. 1974. Mutants of staphylococci with altered cell walls. Ann. N.Y. Acad. Sci. 236:54-62.

64. Patrick, C. C. 1990. Coagulase-negative staphylococci: Pathogens with increasing clinical Significance, J. Pediatr. 116: 497-507.

65. Peacock, S. J., I. de Silva, F. D. Lowy. 2001. What determines nasal carriage of Staphylococcus aureus? Trends Microbiol. 9:605-610.

66. Peiser, L., S. Mukhopadhyay, S. Gordon. 2002. Scavenger receptors in innate immunity. Curr. Opin. Immunol. 14:123-128.

67. Peschel, A., F. Götz. 1996. Analysis of the Staphylococcus epidermidis genes epiF, -E, and -G involved in epidermin immunity. J. Bacteriol. 178:531-536.

68. Peschel, A. et al. 1999. Inactivation of the dlt operon in Staphylococcus aureus confers sensitivity to defensins, protegrins and other antimicrobial peptides. J. Biol. Chem. 274:8405-8410.

69. Peschel, A., et al. 2001. Staphylococcus aureus resistance to human defensins and evasion of neutrophil killing via the novel virulence factor MprF is based on modification of membrane lipids with 1-lysine. J. Exp. Med. 193:1067-1076.

70. Pollack, J. H., F. C. Neuhaus. 1994. Changes in wall teichoic acid during the rod-sphere transition of Bacillus subtilis 168. J. Bacteriol. 176:7252-7259.

71. Pooley, H. M. and Karamata, D. 1994. Teichoic acid synthesis in Bacillus subtilis: genetic organization and biological roles. In Bacterial cell wall. Ghuysen, J.-M. and Hakenbeck, R. (eds). Amsterdam, The Netherlands: Elsevier Science B. V., pp.187-197.

72. Pooley, H. M., Abellan, F. X., and Karamata, D. 1992. CDP-glycerol:poly(glycerophosphate) glycerophosphotransferase, which is involved in the synthesis of the major wall teichoic acid in Bacillus subtilis 168, is encoded by tagF (rodC). J Bacteriol 174:646-649

73. Prince, G A, Jenson, A B, Horswood, R L Carmargo, E, and Chanock, R M. 1978. The Pathogenesis of Respiratory Syncytial Virus Infection in Cotton Rats. Am. J. Pathol. 93:771-783.

74. Ramkissoon-Ganorkar, C. et al. 1999. Modulating insulin-release profile from pH/thermosensivite polymeric beads through polymer molecular weight. J. Contr. Release 59:287-298.

75. Romero-Vivas J.; Rubio M.; Fernandez C.; Picazo J. J. 1995. Mortality associated with nosocomial bacteremia due to methicillin-resistant Staphylococcus aureus, Clin. Infect. Dis. 21: 1417-23.

76. Sambrook, Joseph; Russell, David W. 1989. Molecular Cloning: A Laboratory Manual, 2^{nd} Ed., Cold Spring Harbor Press, Cold Spring Harbor, N.Y.

77. Schägger, H. & G. Jagow. 1987. Tricine-sodium dodecyl sulfate-polyacrylamide gel electrophoresis for the separation of proteins in the range from 1 to 100 kDa. Anal. Biochem. 166:368-379.

78. Schwab, U. E., A. E. Wold, J. L. Carson, M. W. Leigh, P.-W. Cheng, P. H. Gilligan and T. F. Boat. 1993. Increased adherence of Staphylococcus aureus from cystic fibrosis lungs to airway epithelial cells. Am. Rev. Respir. Dis. 148:365-369.

79. Shulman, M., C. D. Wilde and G. Kohler. 1978. A better cell line for making hybridomas secreting specific antibodies. Nature 276:269-270.

80. Smith R. L. and E. Gilkerson. 1979. Quantitation of glycosaminoglycan hexosamine using 3-methyl-2-benzothiazolone hydrazone hydrochloride. Anal. Biochem. 98:478-480.

81. Soane, R. J. et al. 1999. Evaluation of the clearance characteristics of bioadhesive systems in humans. Int. J. Pharm. 178:55-65.

82. Soldo, B., Lazarevic, V. & Karamata, D. 2002. tagO is involved in the synthesis of all anionic cell-wall polymers in Bacillus subtilis 168. Microbiology 148:2079-2087.

83. Soldo, B., Lazarevic, V., Pooley, H. M., and Karamata, D. 2002. Characterization of a Bacillus subtilis thermosensitive teichoic acid-deficient mutant: gene mnaA (yvyH) encodes the UDP-N-acetylglucosamine 2-epimerase. J Bacteriol 184:4316-4320.

84. Soto, N., A. Vaghjimal, A. Stahl-Avicolli, J. Protic, L. Lutwick and E. Chapnick. 1999. Bacitracin versus mupirocin for Staphylococcus aureus nasal colonization. Infect. Cont. Hosp. Epidem. 20:351-353.

85. Suzuki, Y. and Y. Makino. 1999. Mucosal drug delivery using cellulose derivative as a functional polymer. J. Control. Release. 62:101-107.

86. Takeda S.; Pier G. B.; Kojima Y.; Tojo M.; Muller E.; Tosteson T.; Goldmann D. A. 1991. Protection against endocarditis due to Staphylococcus epidermidis by immunization with capsular polysaccharide/adhesin, Circulation 86: 2539-2546.

87. Teti, G., M. S. Chiofalo, F. Tomasello, C. Fava and P. Mastroeni. 1987. Mediation of Staphylococcus saprophyticus adherence to uroepithelial cells by lipoteichoic acid. Infect. Immun. 55:839-842.

88. Timmerman C. P.; Besnier J. M.; De Graaf L.; Torensma R.; Verkley A. J.; Fleer A.; Verhoef J. 1991. Characterisation and functional aspects of monoclonal antibodies specific for surface proteins of coagulase-negative staphylococci, J. Med. Micro. 35: 65-71.

89. Travis, S. M., Singh, P. K. & Welsh, M. J. 2001. Antimicrobial peptides and proteins in the innate defense of the airway surface. Curr. Opin. Immunol.

90. von Eiff, C., K. Becker, K. Machka, H. Stammer, G. Peters. 2001. Nasal carriage as a source of Staphylococcus aureus bacteremia. Study Group. N. Eng. J. Med. 344:11-16.

91. Ward, T. T. 1992. Comparison of in vitro adherence of methicillin-sensitive and methicillin-resistant Staphylococcus aureus to human nasal epithelial cells. J. Infect. Dis. 166:400-404.

92. White, A. and J. Smith. 1963. Nasal reservoir as the source of extranasal staphylococci. Antimicrob. Agent. Chem. 3:679-683.

93. Wolz, C. et al. 2000. Agr-independent regulation of fibronectin-binding protein(s) by the regulatory locus sar in Staphylococcus aureus. Mol. Microbiol. 36:230-243.

94. Yokoyama, Y., Y. Harabuchi, H. Kodama, H. Murakata and A. Kataura. 1996. Systemic immune response to Streptococcal and Staphylococcal lipoteichoic acids in children with recurrent tonsillitis. Acta Otolaryngol. Suppl. (Norway). 523:108-111.

95. Yokoyama, K., Miyashita, T., Araki, Y., and Ito, E. 1986. Structure and functions of linkage unit intermediates in the biosynthesis of ribitol teichoic acids in Staphylococcus aureus H and Bacillus subtilis W23. Eur J Biochem 161:479-489

1. A method for treating a staphylococcal infection in a patient, comprising instilling into the patient a therapeutically effective amount of a composition comprising antibodies or fragments thereof that specifically bind to WTA, wherein treatment results in alleviation or blocking of colonization.

2. The method of claim 1, wherein the composition comprises polyclonal antibodies that specifically bind to WTA.

3. The method of claim 1, wherein the composition comprises a monoclonal antibody that specifically binds to WTA.

4. The method of claim 1, wherein the composition comprises a multiplicity of MAbs that specifically bind WTA, wherein the MAbs have non-identical amino acid sequences.

5. The method of claim 1, wherein the composition comprises a chimeric antibody that specifically binds to WTA.

6. The method of claim 1, wherein the composition comprises a humanized antibody that specifically binds to WTA.

7. The method of claim 1, wherein the composition comprises a human antibody that specifically binds to WTA.

8. The method of claim 1, further comprising the instillation of at least one anti-staphylococcal drug.

9. The method of claim 8, wherein the anti-staphylococcal drug is selected from lysostaphin and nisin.

10. The method of claim 1, wherein the staphylococcal infection is selected from a localized infection, a systemic infection, and a contamination of a foreign body.

11. The method of claim 1, wherein the antibody fragments are chosen from Fab, Fab′, F(ab′)₂, Fv, SFv, and scFv.

12. The method of claim 1, wherein the staphylococcal infection is a S. aureus infection.

13. A method for treating a staphylococcal infection in a patient, comprising instilling into the patient a therapeutically effective amount of a composition comprising a soluble form of whole WTA or a fragment of WTA, wherein treatment results in alleviation or blocking of colonization.

14. The method of claim 13, further comprising the instillation of at least one anti-staphylococcal drug.

15. The method of claim 14, wherein the anti-staphylococcal drug is selected from lysostaphin and nisin.

16. The method of claim 13, wherein the staphylococcal infection is selected from a localized infection, a systemic infection, and a contamination of a foreign body.

17. The method of claim 13, wherein the staphylococcal infection is a S. aureus infection.

18. A composition comprising a therapeutically effective amount of antibodies or fragments thereof that specifically bind to WTA, wherein said antibodies or fragments thereof alleviate or block staphylococcal colonization upon administration to a patient.

19. The composition of claim 18, wherein the composition comprises polyclonal antibodies that specifically bind to WTA.

20. The composition of claim 18, wherein the composition comprises a monoclonal antibody that specifically binds to WTA.

21. The composition of claim 18, wherein the composition comprises a multiplicity of MAbs that specifically bind WTA, wherein the MAbs have non-identical amino acid sequences.

22. The composition of claim 18, wherein the composition comprises a chimeric antibody that specifically binds to WTA.

23. The composition of claim 18, wherein the composition comprises a humanized antibody that specifically binds to WTA.

24. The composition of claim 18, wherein the composition comprises a human antibody that specifically binds to WTA.

25. The composition of claim 18, wherein the fragment is chosen from Fab, Fab′, F(ab′)₂, Fv, SFv, and scFv.

26. The composition of claim 18, wherein the colonization results in a staphylococcal infection selected from a localized infection, a systemic infection, and a contamination of a foreign body.

27. The composition of claim 18, wherein the staphylococcal colonization results in a S. aureus infection.

28. A vaccine comprising:
(a) the composition of claim 18; and
(b) a pharmaceutically acceptable carrier.

29. The vaccine of claim 28, wherein the colonization results in a staphylococcal infection selected from a localized infection, a systemic infection, and a contamination of a foreign body.

30. The vaccine of claim 28, wherein the staphylococcal colonization results in a S. aureus infection.

31. A composition comprising a therapeutically effective amount of a soluble form of whole WTA or fragments thereof, wherein said WTA or fragments thereof alleviate or block staphylococcal colonization upon administration to a patient.

32. The composition of claim 31, wherein the colonization results in a staphylococcal infection selected from a localized infection, a systemic infection, and a contamination of a foreign body.

33. The composition of claim 31, wherein the staphylococcal colonization results in a S. aureus infection.

34. A vaccine comprising:
(a) the composition of claim 31; and
(b) a pharmaceutically acceptable carrier.

35. The vaccine of claim 34, wherein the colonization results in a staphylococcal infection selected from a localized infection, a systemic infection, and a contamination of a foreign body.

36. The vaccine of claim 34, wherein the staphylococcal colonization results in a S. aureus infection.

37. An isolated constructed S. aureus organism deficient in WTA, wherein the tagO gene is inactivated during construction.

38. The isolated S. aureus organism of claim 37, wherein the organism is ΔtagO.

39. The method of claim 1, wherein the instillation is chosen from nasal instillation, oral instillation, airway instillation, and systemic instillation.

40. The method of claim 1, wherein the patient suffers from at least one of cystic fibrosis, staphylococcal pneumonia, a staphylococcal contamination of a foreign body, a staphylococcal infection, and staphylococcal nasal colonization.

41. The method of claim 40, wherein the staphylococcus that causes at least one of cystic fibrosis, staphylococcal pneumonia, a staphylococcal contamination of a foreign body, a staphylococcal infection, and staphylococcal nasal colonization is S. aureus.

42. The method of claim 13, wherein the instillation is chosen from nasal instillation, oral instillation, airway instillation, and systemic instillation.

43. The method of claim 13, wherein the patient suffers from at least one of cystic fibrosis, staphylococcal pneumonia, a staphylococcal contamination of a foreign body, a staphylococcal infection, and staphylococcal nasal colonization.

44. The method of claim 43, wherein the staphylococcus that causes at least one of cystic fibrosis, staphylococcal pneumonia, a staphylococcal contamination of a foreign body, a staphylococcal infection, and staphylococcal nasal colonization is S. aureus.
